(19) United States
(12) Patent Application Publication
(10) Pub. No.: US 2015/0203512 A1
(43) Pub. Date: Jul. 23, 2015

(54) MACROCYCLIC COMPOUNDS AS HIV INTEGRASE INHIBITORS

(71) Applicant: MERCK SHARP & DOHME CORP., Rahway, NJ (US)

(72) Inventors: Thomas S. Reger, Lansdale, PA (US); Abbas M. Walji, Lansdale, PA (US); John M. Sanders, Hatfield, PA (US); John S. Wai, Harleysville, PA (US); Liping Wang, Shanghai (CN)

(21) Appl. No.: 14/414,272

(22) PCT Filed: Jul. 10, 2013
(86) PCT No.: PCT/US2013/049922

§ 371 Date: Jan. 12, 2015

(30) Foreign Application Priority Data
Jul. 11, 2012 (CN) PCT/CN2012/78465

(51) Int. Cl. C07D 498/18; A61K 31/553; A61K 45/06; A61K 31/55

(52) U.S. Cl. CPC C07D 498/18; A61K 31/55; A61K 31/553; A61K 45/06

(57) ABSTRACT

The present invention relates to Macrocyclic Compounds. The present invention also relates to compositions comprising at least one Macrocyclic Compound, and methods of using the Macrocyclic Compounds for treating or preventing HIV infection in a subject.

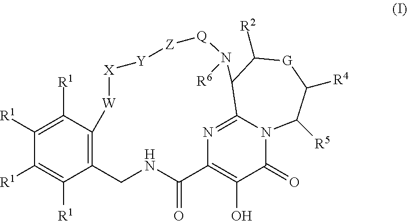

### FIELD OF THE INVENTION

The present invention relates to Macrocyclic Compounds, compositions comprising at least one Macrocyclic Compound, and methods of using the Macrocyclic Compounds for treating or preventing HIV infection in a subject.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)] Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells.

The following references are of interest as background:

International Publication Nos. WO 11/045330 and WO 11/121105 disclose macrocyclic compounds having HIV integrase inhibitory activity.

Kinzel et al., Tet. Letters 2007, 48(37): pp. 6552-6555 discloses the synthesis of tetrahydropyridopyrimidones as a scaffold for HIV-1 integrase inhibitors.

Ferrara et al., Tet. Letters 2007, 48(37), pp. 8379-8382 discloses the synthesis of a hexahydropyrimido[1,2-a]azepine-2-carboxamide derivative useful as an HIV integrase inhibitor.

Muraglia et al., J. Med. Chem. 2008, 51: 861-874 discloses the design and synthesis of bicyclic pyrimidinones as potent and orally bioavailable HIV-1 integrase inhibitors.

US2004/229909 discloses certain compounds having integrase inhibitory activity.

U.S. Pat. No. 7,232,819 and US 2007/0083045 disclose certain 5,6-dihydroxypyrimidine-4-carboxamides as HIV integrase inhibitors.

U.S. Pat. No. 7,169,780, U.S. Pat. No. 7,217,713, and US 2007/0123524 disclose certain N-substituted 5-hydroxy-6-oxo-1,6-dihydropyrimidine-4-carboxamides as HIV integrase inhibitors.

U.S. Pat. No. 7,279,487 discloses certain hydroxynaphthyridinone carboxamides that are useful as HIV integrase inhibitors.

U.S. Pat. No. 7,135,467 and U.S. Pat. No. 7,037,908 disclose certain pyrimidine carboxamides that are useful as HIV integrase inhibitors.

U.S. Pat. No. 7,211,572 discloses certain nitrogenous condensed ring compounds that are HIV integrase inhibitors.

U.S. Pat. No. 7,414,045 discloses certain tetrahydro-4H-pyrido[1,2-a]pyrimidine carboxamides, hexahydropyrimido[1,2-a]azepine carboxamides, and related compounds that are useful as HIV integrase inhibitors.

WO 2006/103399 discloses certain tetrahydro-4H-pyrimidooxazepine carboaxmides, tetrahydropyrazinopyrimidine carboxamides, hexahydropyrimidodiazepine carboxamides, and related compounds that are useful as HIV integrase inhibitors.

US 2007/0142635 discloses processes for preparing hexahydropyrimido[1,2-a]azepine-2-carboxylates and related compounds.

US 2007/0149556 discloses certain hydroxypyrimidinone derivatives having HIV integrase inhibitory activity.

Various pyrimidinone compounds useful as HIV integrase inhibitors are also disclosed in U.S. Pat. No. 7,115,601, U.S. Pat. No. 7,157,447, U.S. Pat. No. 7,173,022, U.S. Pat. No. 7,176,196, U.S. Pat. No. 7,192,948, U.S. Pat. No. 7,273,859, and U.S. Pat. No. 7,419,969.

US 2007/0111984 discloses a series of bicyclic pyrimidinone compounds useful as HIV integrase inhibitors.

US 2006/0276466, US 2007/0049606, US 2007/0111985, US 2007/0112190, US 2007/0281917, US 2008/0004265 each disclose a series of bicyclic pyrimidinone compounds useful as HIV integrase inhibitors.

### SUMMARY OF THE INVENTION

In one aspect, the present invention provides Compounds of Formula (I):

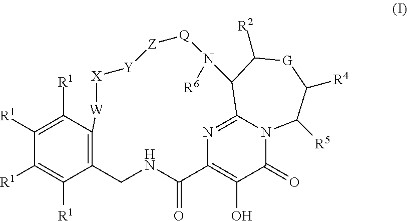

and pharmaceutically acceptable salts and prodrugs thereof,

wherein:
G is —O— or —CH(R³)—;
Q is —C(O)— or —S(O)₂—;
W is a bond, —O— or —N(R⁸)—;
X is a bond or —C(O)—;
Y is C₃-C₅ alkylene or C₃-C₅ alkenylene;
Z is a bond, —O— or —N(R⁸)C(O)—;
each occurrence of R¹ is independently selected from H, C₁-C₆ alkyl, halo, C₁-C₆ haloalkyl, 3 to 7-membered cycloalkyl, —OR⁷, —N(R⁷)₂, —CN, —C(O)R⁷, —C(O)OR⁷, —C(O)N(R⁷)₂ and —NHC(O)R⁷;
R², R³, R⁴ and R⁵ are each independently selected from H, C₁-C₆ alkyl, halo, —OR⁷ and —N(R)₂;
R⁶ is H or C₁-C₆ alkyl, 3 to 7-membered cycloalkyl, C₆-C₁₀ aryl, 4 to 7-membered monocyclic heterocycloalkyl or 5 or 6-membered monocyclic heteroaryl;
each occurrence of R⁷ is independently selected from C₁-C₆ alkyl, 3 to 7-membered cycloalkyl, C₆-C₁₀ aryl, 4 to 7-membered monocyclic heterocycloalkyl or 5 or 6-membered monocyclic heteroaryl; and each occurrence of R⁸ is independently selected from H and C₁-C₆ alkyl.

The Compounds of Formula (I) (also referred to herein as the “Macrocyclic Compounds”) and pharmaceutically acceptable salts thereof can be useful, for example, for inhibiting HIV viral replication or replicon activity, and for treating or preventing HIV infection in a subject. Without being bound by any specific theory, it is believed that the Macrocyclic Compounds inhibit HIV viral replication by inhibiting HIV Integrase.

Accordingly, the present invention provides methods for treating or preventing HIV infection in a subject, comprising administering to the subject an effective amount of at least one Macrocyclic Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to Macrocyclic Compounds, compositions comprising at least one Macrocyclic Compound, and methods of using the Macrocyclic Compounds for treating or preventing HIV infection in a subject.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . . .

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

The term “effective amount” as used herein, refers to an amount of Macrocyclic Compound and/or an additional therapeutic agent, or a composition thereof that is effective in producing the desired therapeutic, ameliorative, inhibitory or preventative effect when administered to a subject suffering from HIV infection or AIDS. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HIV viral infection or AIDS, refers to reducing the likelihood or severity of HIV infection or AIDS.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)— alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms. The term “C₃-C₅ alkylene” refers to an alkylene group having from 3 to 5 carbon atoms.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH═CHCH₂—, —CH₂CH═CH—, —CH₂CH═CHCH₂—, —CH═CHCH₂CH₂—, —CH₂CH₂CH═CH— and —CH(CH₃)CH═CH—. In one embodiment, an alkenylene group has from 2 to about 6 carbon atoms. In another embodiment, an alkenylene group has from about 3 to about 5 carbon atoms. In another embodiment, an alkenylene group is branched. In another embodiment, an alkenylene group is linear. The term “C₂-C₆ alkylene” refers to an alkenylene group having from 2 to 6 carbon atoms. The term “C₃-C₅ alkenylene” refers to an alkenylene group having from 3 to 5 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

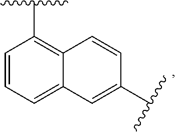 [SMILES: *c1ccc2c(*)cccc2c1]

is understood to represent both:

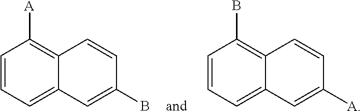

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

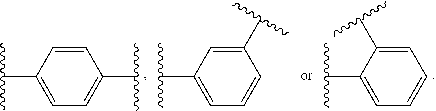

Unless otherwise indicated, an arylene group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

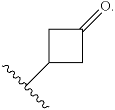 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered monocyclic heteroaryl. In another embodiment, a heteroaryl group comprises a 5- to 6-membered monocyclic heteroaryl group fused to a benzene ring. Unless otherwise indicated, a heteroaryl group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone and the like, and all isomers thereof.

A ring carbon atom of a heterocycloalkyl group may be functionalized as a carbonyl group. An illustrative example of such a heterocycloalkyl group is:

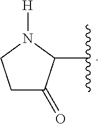 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic heterocycloalkyl” refers to a monocyclic heterocycloalkyl group having from 3 to 6 ring atoms. The term “4 to 7-membered monocyclic heterocycloalkyl” refers to a monocyclic heterocycloalkyl group having from 4 to 7 ring atoms. The term “7 to 11-membered bicyclic heterocycloalkyl” refers to a bicyclic heterocycloalkyl group having from 7 to 11 ring atoms. Unless otherwise indicated, a heterocycloalkyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O— alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

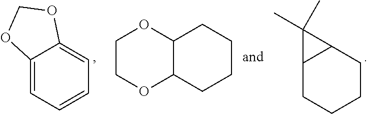

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R¹, R⁷, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Macrocyclic Compound or a pharmaceutically acceptable salt of the compound. The transformation may occur by various mechanisms (e.g., by metabolic or chemical processes), such as, for example, through hydrolysis in blood. For example, if a Macrocyclic Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Macrocyclic Compound contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a Macrocyclic Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Macrocyclic Compounds can form salts which are also within the scope of this invention. Reference to a Macrocyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Macrocyclic Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Macrocyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Macrocyclic Compounds may be atropisomers (e.g., substituted biaryls) and are considered as part of this invention. Enantiomers can also be directly separated using chiral chromatographic techniques.

It is also possible that the Macrocyclic Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Macrocyclic Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Macrocyclic Compounds, and of the salts, solvates, hydrates, esters and prodrugs of the Macrocyclic Compounds, are intended to be included in the present invention.

The following abbreviations are used below and have the following meanings: AcOH is acetic acid; n-BuLi is n-butyllithium; m-CPBA is 3-chloroperoxybenzoic acid; DABCO is 1,4-diazabicyclo(2,2,2)octane; DEA is diethylamine; DIPEA is N,Ndiisopropylethylamine; DMA is dimethylacetamide; DMF is dimethylformamide; EDCI is 1-ethyl-3-(3′-dimethylaminopropyl)carbodiimide hydrochloride; ESI is electrospray ionization; EtOAc isethyl acetate; EtOH is ethanol; HATU is 2-(1H-7-Azabenzotriazol-1-yl)-1,1,3,3-tetramethyl uronium hexafluorophosphate; HOAt is 1-hydroxy-7-azabenzotriazole; HPLC is high-pressure liquid chromatography; IPA is isopropanol; IPAc is iso-propyl acetate; KOt-Bu is potassium tert-butoxide; LCMS is liquid chromatography-mass spectrometry; LiHMDS is lithium hexamethyldisilylazide; MeCN is acetonitrile; MeOH is methanol; Ms is mesyl or methanesulfonyl; MS is mass spectroscopy; MTBE is methyl tert-butyl ether; NHS is normal human serum; NMR is nuclear magnetic resonance spectroscopy; Piv is pivalate or 2,2-dimethylpropanoyl; Pd/C is palladium on carbon; PyClu is 1-(chloro-1-pyrrolidinylmethylene) pyrrolidinium hexafluorophosphate; SFC is supercritical fluid chromatography; TBAF is n-tetrabutylammonium fluoride; TFA is trifluoroacetic acid; TLC is thin-layer chromatography; Ts is tosyl or 4-toluenesulfonyl; THF is tetrahydrofuran; and Zhan-1b is N-dimethylaminosulfonyl)phenyl]methyleneruthenium(II) dichloride.

### The Compounds of Formula (I)

The present invention provides Macrocyclic Compounds of Formula (I):

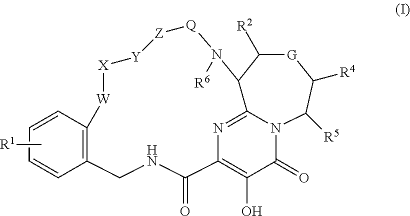

and pharmaceutically acceptable salts thereof, wherein G, Q, W, X, Y, Z, R¹, R², R³, R⁴, R⁵ and R⁶ are defined above for the Compounds of Formula (I).
e

In one embodiment, G is —O—.

In another embodiment, G is —CH(R³)—.

In another embodiment, G is —CH₂—.

In one embodiment, Q is —C(O)—.

In another embodiment, Q is —S(O)₂—.

In one embodiment, W is —O—.

In another embodiment, W is a bond.

In another embodiment, W is —N(R⁸)—.

In still another embodiment, W is —NH—.

In one embodiment, X is a bond.

In another embodiment, X is —C(O)—.

In one embodiment, Y is C₃-C₅ alkylene.

In another embodiment, Y is C₃-C₅ alkenylene.

In another embodiment, Y is —CH₂CH₂CH₂—.

In still another embodiment, Y is —CH₂CH₂CH₂CH₂—.

In another embodiment, Y is —CH₂CH₂CH₂CH₂CH₂—.

In another embodiment, Y is —CH₂CH₂CH═CH—.

In yet another embodiment, Y is —CH₂CH═CHCH₂—.

In a further embodiment, Y is:

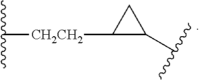

In one embodiment, Z is a bond.

In another embodiment, Z is —C(O)—.

In another embodiment, Z is a —N(R⁸)C(O)—

In another embodiment, Z is —NHC(O)—.

In one embodiment, R¹ represents a single ring substituent, selected from C₁-C₆ alkyl, halo, C₁-C₆ haloalkyl, 3 to 7-membered cycloalkyl, —OR⁷, —N(R⁷)₂, —CN, —C(O)R⁷, —C(O)OR⁷, —C(O)N(R⁷)₂ and —NHC(O)R⁷.

In another embodiment, R¹ represents a single halo substituent.

In another embodiment, R¹ represents a single F substituent.

In still another embodiment, R¹ represents two ring substituents, each independently selected from C₁-C₆ alkyl, halo, C₁-C₆ haloalkyl, 3 to 7-membered cycloalkyl, —OR⁷, —N(R⁷)₂, —CN, —C(O)R⁷, —C(O)OR⁷, —C(O)N(R⁷)₂ and —NHC(O)R⁷.

In another embodiment, R¹ represents two ring substituents, each independently selected from C₁-C₆ alkyl and halo.

In yet another embodiment, R¹ represents two ring substituents, each independently selected from F, Cl and methyl.

In one embodiment R² is H.

In one embodiment R⁴ is H or —O—(C₁-C₆ alkyl).

In another embodiment R⁴ is H or methoxy.

In one embodiment R⁵ is H or C₁-C₆ alkyl.

In another embodiment, R⁵ is H or ethyl.

In one embodiment, R² is H, R⁴ is H or methoxy and R⁵ is H or ethyl.

In one embodiment, G is —CH₂—, and R², R⁴ and R⁵ are each H.

In another embodiment, G is —O—, and R² and R⁴ are each H and R⁵ is ethyl.

In one embodiment R⁶ is C₁-C₆ alkyl.

In another embodiment R⁶ is methyl.

In one embodiment, the compounds of formula (I) have the formula (Ia):

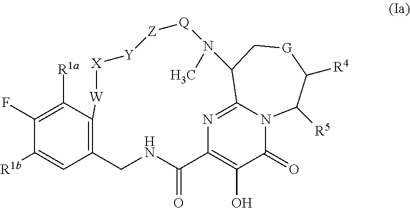

wherein:

Q is —C(O)— or —S(O)₂—;

W is a bond, —O—, —NH— or —N(C₁-C₆ alkyl)-;

X is a bond or —C(O)—;

Y is C₃-C₅ alkylene or C₃-C₅ alkenylene, wherein any two adjacent carbon atoms of a C₃-C₅ alkylene group may be joined by a methylene group to form a cyclopropyl ring;

Z is a bond, —O— or —N(CH₃)C(O)—;

R^{1a} is H or Cl;

R^{1b} is H, C₁-C₆ alkyl or Cl;

R⁴ is H or —O—(C₁-C₆ alkyl); and

R⁵ is H or C₁-C₆ alkyl.

In one embodiment, for the compounds of formula (Ia), R^{1b} is H, methyl or Cl; R⁴ is H or methoxy and R⁵ is H or ethyl.

In another embodiment, for the compounds of formula (Ia), R^{1b} is H; R⁴ is H and R⁵ is ethyl.

In another embodiment, for the compounds of formula (Ia), Q is —C(O)—.

In still another embodiment, for the compounds of formula (Ia), Y is —CH₂CH₂CH₂CH₂—, —CH₂CH₂CH═CH—, CH₂CH═CHCH₂— or:

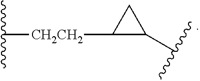

In another embodiment, for the compounds of formula (Ia), Z is a bond.

In yet another embodiment, In another embodiment, for the compounds of formula (Ia), R^{1b} is H; R⁴ is H; R⁵ is ethyl; G is —O—; Q is —C(O)—; Y is —CH₂CH₂CH₂CH₂—, —CH₂CH₂CH═CH—, CH₂CH═CHCH₂— or:

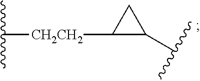

and Z is a bond.

In a further embodiment, for the compounds of formula (Ia), R^{1b} is H; R⁴ is H or methoxy; R⁵ is H; G is —CH—; Q is —C(O)—; Y is —CH₂CH₂CH₂CH₂—, —CH₂CH₂CH═CH—, CH₂CH═CHCH₂— or:

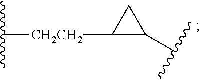

and Z is a bond.

In one embodiment, the compounds of formula (I) have the formula (Ib):

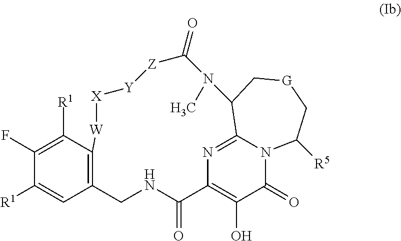

wherein:

G is —O— or —CH₂—

W is a bond, —O— or —N(R⁸)—;

X is a bond or —C(O)—;

Y is C₃-C₅ alkylene or C₃-C₅ alkenylene;

Z is a bond, —O— or —N(R⁸)C(O)—;

R¹ is H or halo; and

R⁵ is H or C₁-C₆ alkyl.

In one embodiment, variables G, Q, W, X, Y, Z, R¹, R², R³, R⁴, R⁵ and R⁶ for the Compounds of Formula (I) are selected independently of each other.

In another embodiment, the Compounds of Formula (I) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HIV antiviral agents, immunomodulators, and anti-infective agents.

(c) The pharmaceutical composition of (b), wherein the HIV antiviral agent is an antiviral selected from the group consisting of HIV protease inhibitors, HIV integrase inhibitors, nucleoside reverse transcriptase inhibitors and non-nucleoside reverse-transcriptase inhibitors.

(d) A pharmaceutical combination that is (i) a Compound of Formula (I) and (ii) a second therapeutic agent selected from the group consisting of HIV antiviral agents, immunomodulators, and anti-infective agents; wherein the Compound of Formula (I) and the second therapeutic agent are each employed in an amount that renders the combination effective for inhibiting HIV replication, or for treating HIV infection and/or reducing the likelihood or severity of symptoms of HIV infection.

(e) The combination of (d), wherein the HIV antiviral agent is an antiviral selected from the group consisting of HIV protease inhibitors, HIV integrase inhibitors, nucleoside reverse transcriptase inhibitors and non-nucleoside reverse-transcriptase inhibitors.

(f) A method of inhibiting HIV replication in a subject in need thereof which comprises administering to the subject an effective amount of a Compound of Formula (I).

(g) A method of treating HIV infection and/or reducing the likelihood or severity of symptoms of HIV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HIV antiviral agents, immunomodulators, and anti-infective agents.

(i) The method of (h), wherein the HIV antiviral agent is an antiviral selected from the group consisting of HIV protease inhibitors, HIV integrase inhibitors, nucleoside reverse transcriptase inhibitors and non-nucleoside reverse-transcriptase inhibitors.

(j) A method of inhibiting HIV replication in a subject in need thereof which comprises administering to the subject the pharmaceutical composition of (a), (b) or (c) or the combination of (d) or (e).

(k) A method of treating HIV infection and/or reducing the likelihood or severity of symptoms of HIV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine, (b) inhibiting HIV replication or (c) treating HIV infection and/or reducing the likelihood or severity of symptoms of HIV infection. In these uses, the compounds of the present invention can optionally be employed in combination with one or more second therapeutic agents selected from HIV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Non-limiting examples of the Compounds of Formula (I) include compounds 1-28 as set forth below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes A-E below. Alternative synthetic pathways and analogous structures will be apparent to those skilled in the art of organic synthesis.

Scheme A describes a method useful for making compounds of Formula (I) wherein a bis-olefin of formula A-1 can undergo a ring-closing metathesis in the presence of a ruthenium catalyst (e.g. Grubbs II or Zhan-1b) to generate a macrocycle of formula A-2. A sulfonate protecting group on A-2 (Pg¹=SO₂Me or SO₂Ph) is then readily removed by treatment with a base such as LiOH or NaOH to provide intermediates of formula A-3. The double-bond present in A-3 can be reduced under an atmosphere of hydrogen in the presence of a heterogeneous catalyst such as Pd/C to provide compounds of formula A-4. This method is also useful for cyclizing intermediate olefin compounds such as carbamates of formula A-5 and oxalamides of formula A-6.

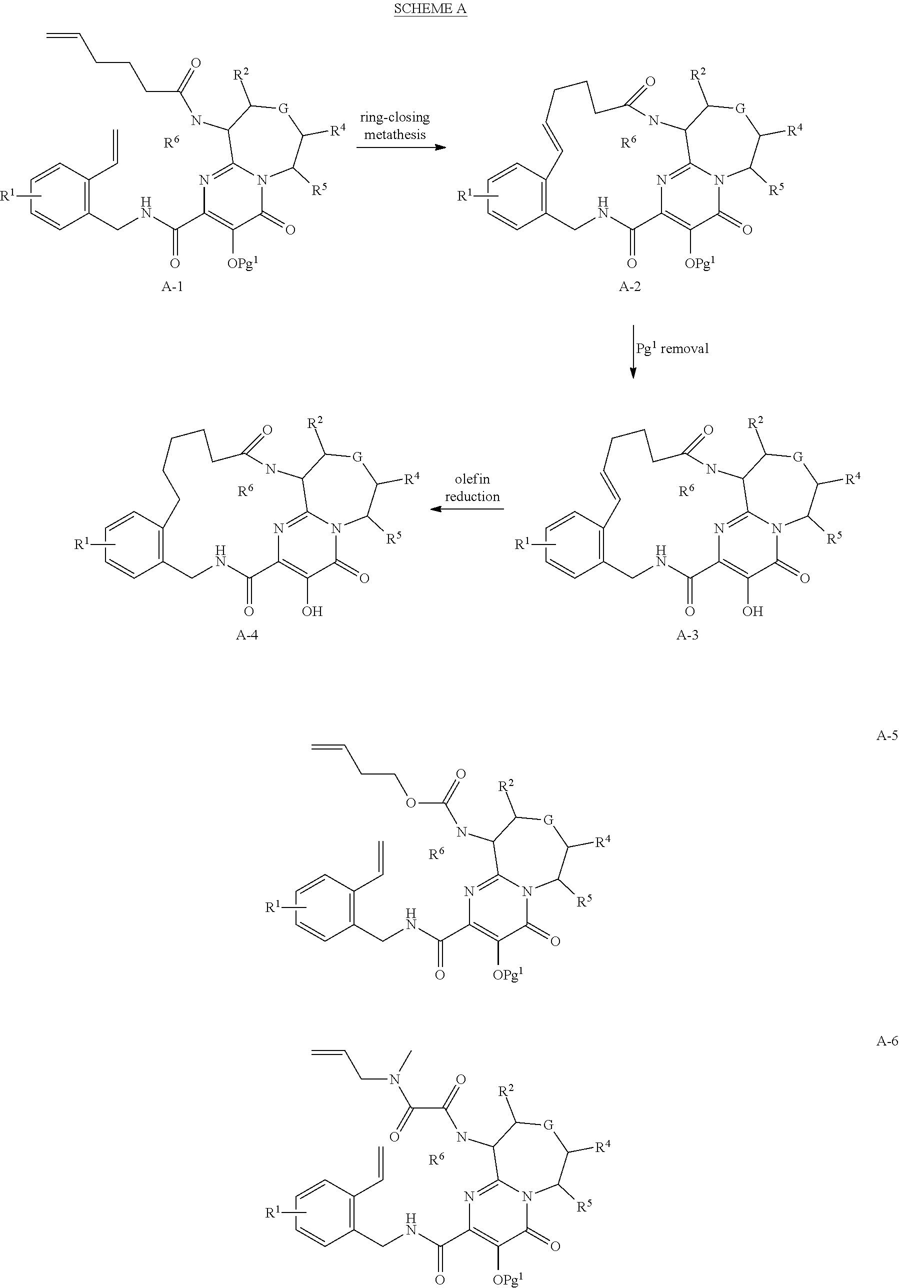

Scheme B sets forth a method useful for making the starting materials of formulas A-1, A-5, and A-6 shown in Scheme A. Esters of formula B-1 (R^{x}=Me, Et; Pg¹=SO₂Me or SO₂Ph; Pg²=Boc) have been described previously (WO 2005/061501 A2 and US 2010/0087419 A1) and can undergo reaction with benzylamines that have been pre-mixed with trimethylaluminum to provide the trans-amidation products of formula B-2. In some cases, an appropriate benzylamine can react thermally (e.g. 70-150° C. in a microwave reactor) in a solvent such as ethanol to provide the compounds of formula B-2. The aryl bromide in B-2 can then be transformed to an alkene via cross-coupling with a vinyl-metal species such as tributylvinyltin or potassium vinyltrifluoroborate in the presence of a Pd catalyst (e.g, Pd(PPh₃)₄ or Pd(PPh₃)₂Cl₂). The amine protecting group (Pg²=Boc) can be removed by treatment with a strong acid such as HCl or TFA to provide the amino compounds of formula B-4. The secondary amine in B-4 can then reacted with a carboxylic acid in the presence of suitable coupling reagents (e.e EDC/HOAt, HATU, or CDI) to provide the amides of formula A-1. Alternatively, the amine can react with an acid chloride in the presence of a base (e.g. triethylamine or pyridine) to provide the compounds of formula A-1. In a similar manner, B-4 can react with alkyl chloroformates and carbonates to provide carbamates of formula A-5 and with oxamic acids to provide oxalamides of formula A-6.

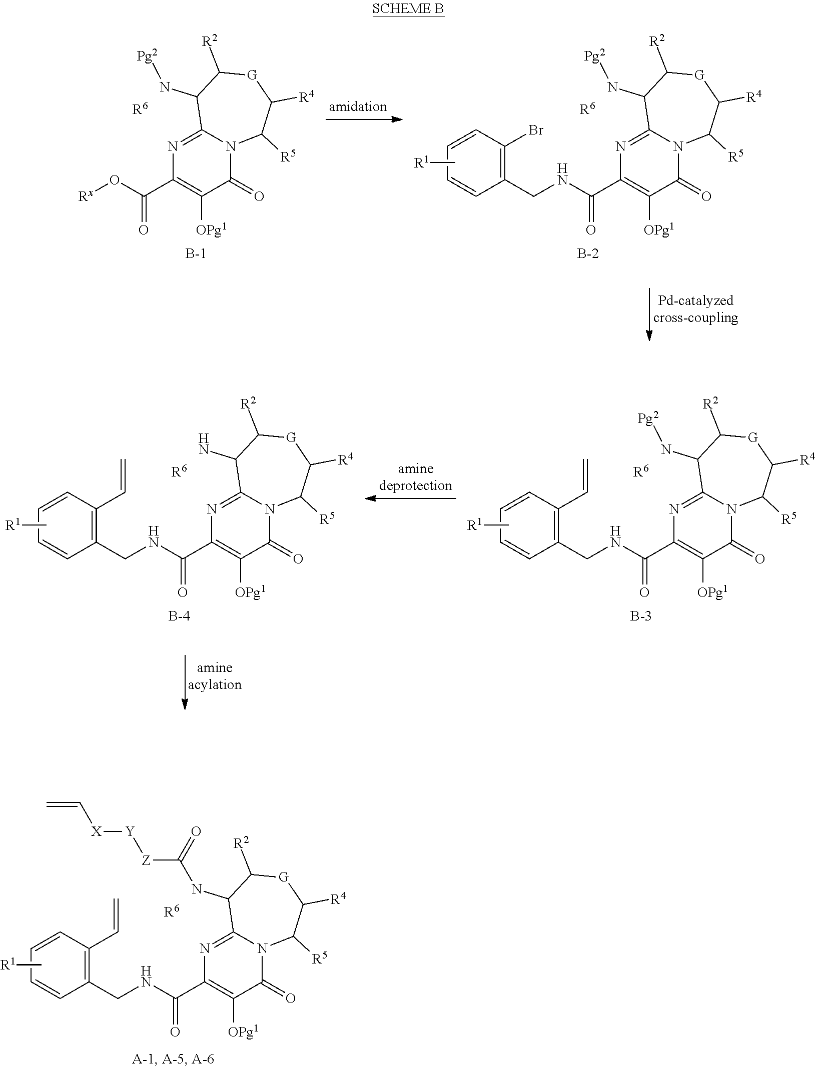

Scheme C illustrates an alternate method useful for preparation of compounds of Formula (I) whereby a carboxylic acid of formula C-1 (described, for example, in U.S. Patent Publication No. US 2010/0087419) containing a protecting group (Pg¹=SO₂Me or SO₂Ph) and a leaving group (i.e. LG=Br or OMs) is reacted with a primary benzylamine such as C-2 (W═O or N—R) in the presence of a coupling reagent such as PyClu or HATU and a hindered base (e.g. triethylamine or Hunig's base) to provide compounds of formula C-3. The leaving group (LG) can then be substituted by a primary amine (e.g. methylamine or ethylamine) to provide compounds of formula C-4. Subsequent acylation with acryloyl chloride and ring-closing metathesis with a ruthenium catalyst as described in Scheme A provides macrocycles of formula C-6. Olefin reduction as previously described in Scheme A, provides the compounds of formula C-7. Alternatively, alkenes of formula C-6 can react with a reagent such as trimethylsulfoxonium iodide in the presence of a strong base such as sodium hydride or sodium tert-butoxide to provide cyclopropane compounds of formula C-8.

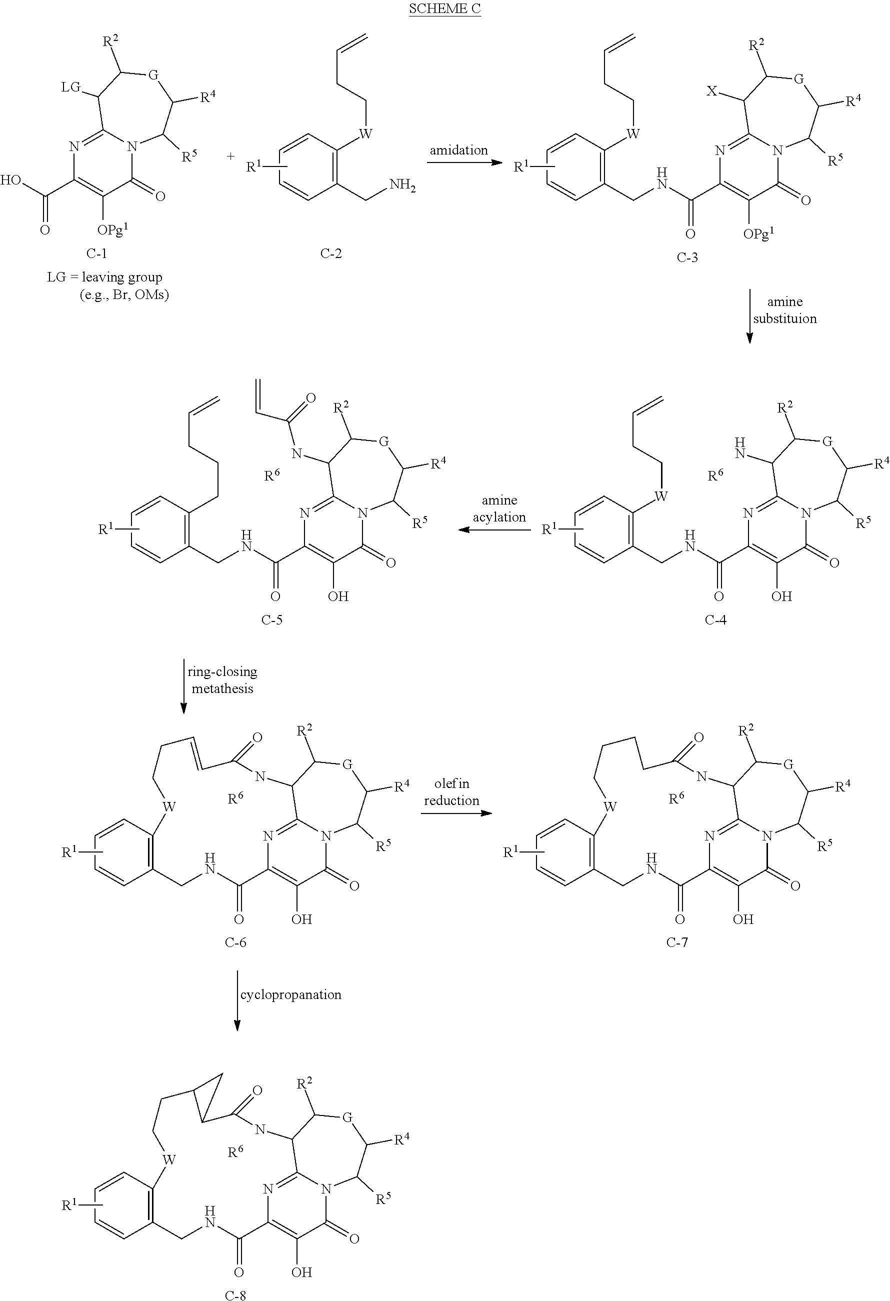

Scheme D describes a method useful for the incorporation of a sulfonamide functional group as part of the macrocycle linker. A compound of formula C-1 can undergo amidation, followed by amine displacement of an appropriate leaving group (e.g., LG=OMs or Br) to provide intermediate alcohols of formula D-3. Reaction of the pendant amine with a sulfonyl chloride in the presence of triethylamine or Hunig's base generates the macrocyclization precursors of formula D-4. Ring-closing metathesis of D-4 to provide the compounds of formula D-5, followed by olefin reduction as described in Scheme A provides the sulfonamides of formula D-6.

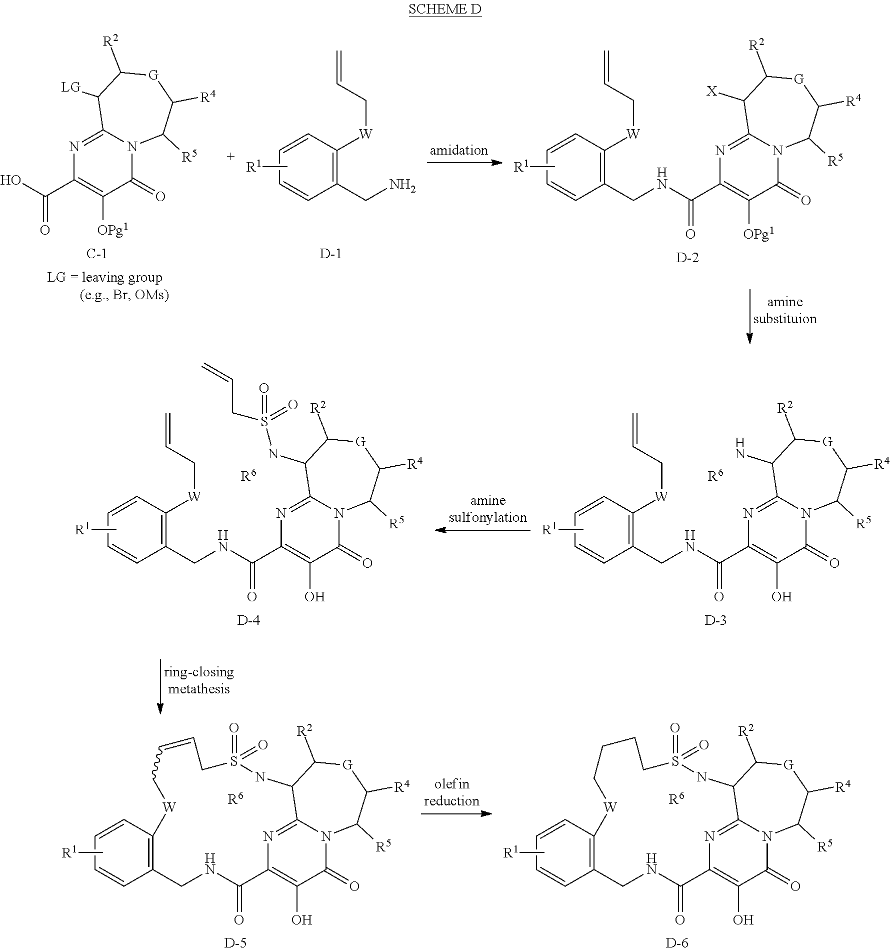

Scheme E describes a method useful for making compounds of formula E-6 which correspond to the compounds of Formula (I) having multiple amide groups in the macrocycle tether.

A compound of formula B-1 can be reacted with a compound of formula E-1 in a microwave reactor in a polar solvent such as ethanol to provide the coupled compounds of formula E-2. Reaction of E-2 with a dicarbonyl compound of formula E-3 (R^{y}═Cl, R^{z}=Me) in the presence of a tertiary amine base provides compounds of formula E-4. Alternatively, a reagent of formula E-3 (R^{y}═OH, R^{z}=tBu) can be reacted with a compound of formula E-2 in the presence of a coupling reagent (e.g. HATU or PyClu) to provide compounds of formula E-4. For compounds of formula E-4, where R^{z}=tBu, ester deprotection and amine deprotection (Pg²=Boc) can be accomplished using a strong acid such as TFA or HCl to provide the amines of formula E-5. In the case where R^{z}=Me, a two-step deprotection process consisting of basic ester hydrolysis (aqueous LiOH or NaOH) followed by Boc removal (TFA or HCl) provides the compounds of formula E-5. Compounds of formula E-5 can then undergo intramolecular macrolactam formation to provide the compounds of formula E-6 using a dehydrating agent such as PyClu, HATU, or HBTU.

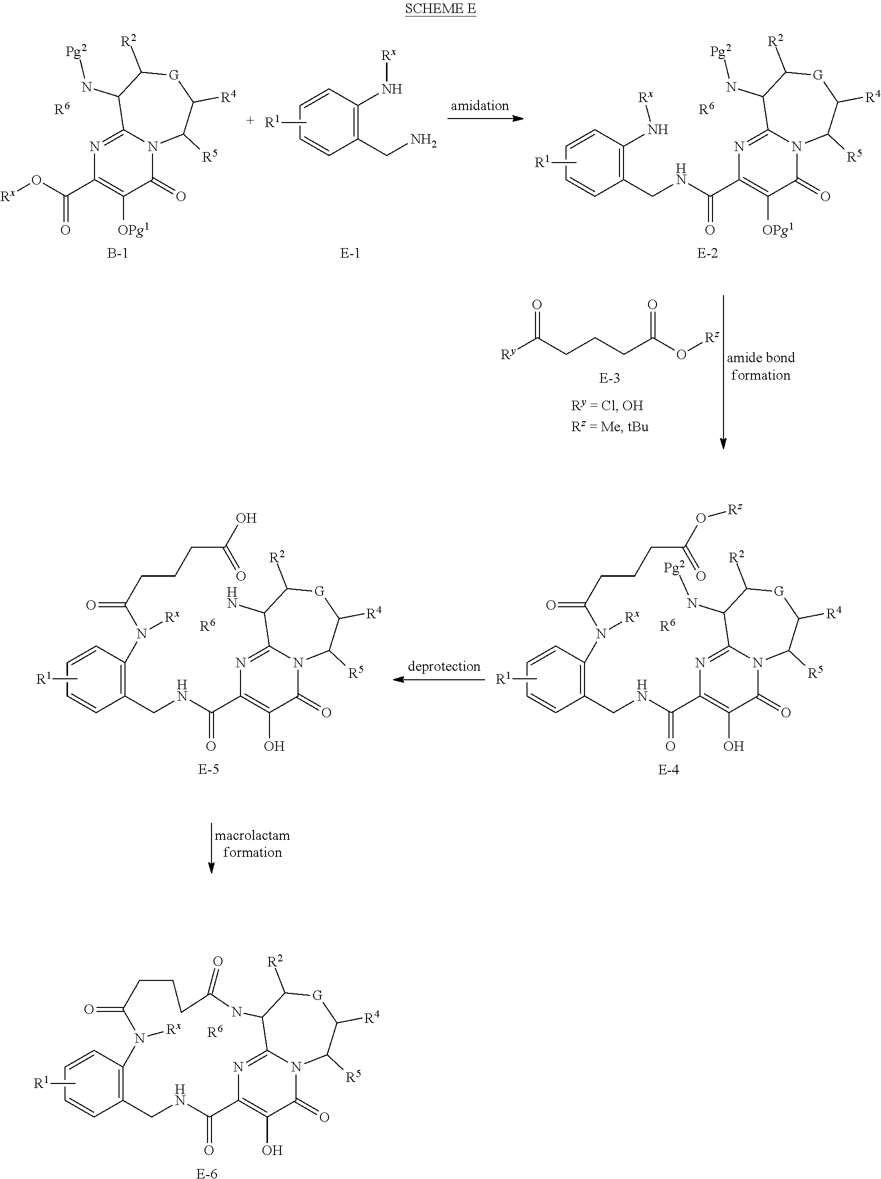

One skilled in the art of organic synthesis will recognize that the synthesis of compounds with multiple reactive functional groups, such as —OH and NH₂, may require protection of certain functional groups (i.e., derivatization for the purpose of chemical compatibility with a particular reaction condition). Suitable protecting groups for the various functional groups of these compounds and methods for their installation and removal are well-known in the art of organic chemistry. A summary of many of these methods can be found in Greene & Wuts, Protecting Groups in Organic Synthesis, John Wiley & Sons, 3^{rd} edition (1999).

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

Compounds of formula C-6, C-7, C-8, D-5, D-6 and E-6 may be further elaborated using methods that would be well-known to those skilled in the art of organic synthesis or, for example, the methods described in the Examples below, to make the full scope of the Compounds of Formula (I).

The starting materials used and the intermediates prepared using the methods set forth in Schemes A-E may be isolated and purified if desired using conventional techniques, including but not limited to filtration, distillation, crystallization, chromatography and alike. Such materials can be characterized using conventional means, including physical constants and spectral data.

### EXAMPLES

### General Methods

Solvents, reagents, and intermediates that are commercially available were used as received. Reagents and intermediates that are not commercially available were prepared in the manner described below. Compounds were purified using either 1) reverse-phase HPLC using acetonitrile/water mixtures as eluent, 2) preparative-TLC using dichloromethane/MeOH or EtOAc/hexane as the mobile phase or 3) flash silica gel chromatography using dichloromethane/MeOH or EtOAc/hexane as the mobile phase. Specific purification conditions are given in the experimental procedures. ¹H NMR spectra were recorded on Varian or Bruker instruments 400-500 MHz and signals are reported as ppm downfield from Me₄Si with number of protons, multiplicities, and coupling constants in Hertz indicated parenthetically.

### Example 1

### Preparation of Compound Int-1d

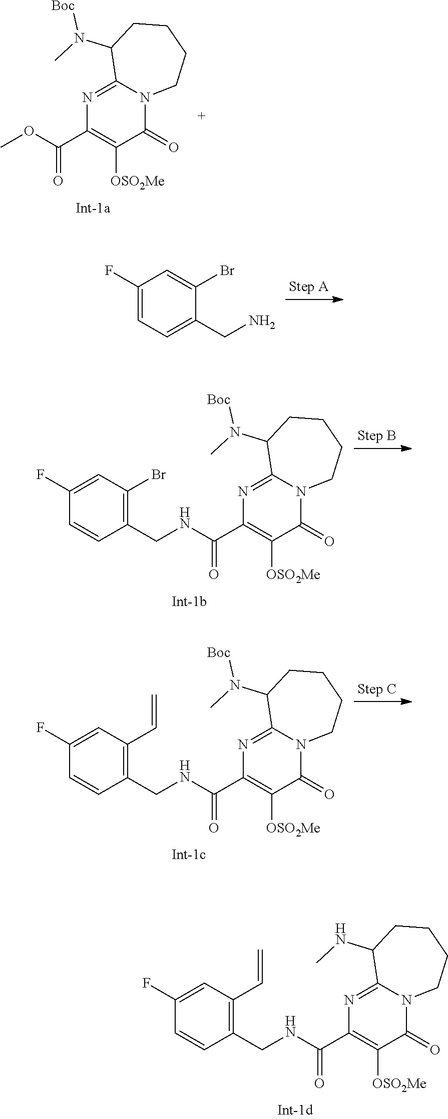

### Step A—Synthesis of Intermediate Compound Int-1b

To a solution of 2-bromo-4-fluorobenzylamine (820 mg, 4 mmol) in dichloromethane (5 mL) was added a solution of AlMe₃ (2 mL, 2.0 M in toluene, 4 mmol) at −5° C. and the reaction mixture was allowed to stir at the same temperature for 15 minutes. A solution of compound Int-1a (previously described in WO2005061501A2) (1.0 g, 2.7 mmol) in dichloromethane (5 mL) was added at −5° C. and the reaction mixture was then stirred at room temperature for about 15 hours. The mixture was cooled to 5° C. then carefully quenched with water and extracted with EtOAc. The combined organic layers were washed with brine, dried over Na₂SO₄ and concentrated in vacuo. The crude residue was purified using prep-TLC (30:1 dichloromethane:MeOH) to provide compound Int-1b (1.2 g, 90%). ¹H NMR (400 MHz, CDCl₃) δ 7.85-7.86 (m, 1H), 7.46-7.47 (m, 1H), 7.30-7.32 (m, 1H), 6.99-7.02 (m, 1H), 5.35-5.37 (m, 1H), 5.20-5.23 (m, 1H), 4.61-4.64 (m, 2H), 3.54 (s, 3H), 2.94 (s, 3H), 2.09-2.11 (m, 3H), 1.85-1.88 (m, 1H), 1.71-1.74 (m, 1H), 1.36-1.41 (m, 2H), 1.20 (s, 9H). LCMS anal. calcd. For C₂₄H₃₀BrFN₄O₂S: 616.1. Found: 616.2 (M⁺).

### Step B—Synthesis of Intermediate Compound Int-1c

To a mixture of compound Int-1b (593 mg, 1.0 mmol) and tributyl-vinylstannane (1.1 g, 3.0 mmol) in toluene (10 mL) was added Pd(PPh₃)₄ (115 mg, 0.1 mmol) at room temperature, and then the mixture was heated to reflux for about 15 hours. After cooling to room temperature, the mixture was diluted with water and extracted with EtOAc. The combined organic layers were washed with brine, dried over Na₂SO₄ and concentrated in vacuo. The crude residue was purified using prep-TLC (30:1 dichloromethane:MeOH) to provide compound Int-1c (500 mg, 88%). LCMS anal. calcd. for C₂₆H₃₃FN₄O₇S: 564.2. Found: 565.1 (M+1)⁺.

Step C—Synthesis of Intermediate Compound Int-1d To a solution of compound Int-1c (500 mg, 0.89 mmol) in MeOH (5 mL) was added a saturated solution of HCl in MeOH (10 mL). The mixture was allowed to stir at room temperature for 1 hour, and then the reaction was adjusted to pH 7 by addition of Et₃N. The reaction mixture was concentrated in vacuo (bath temp <25° C.) to provide compound Int-1d (455 mg, yield: 100%) as a white solid. LCMS anal. calcd. for C₂₁H₂₅FN₄O₅S: 464.1. Found: 465.1 (M+1)⁺.

### Example 2

### Preparation of Compound Int-2b

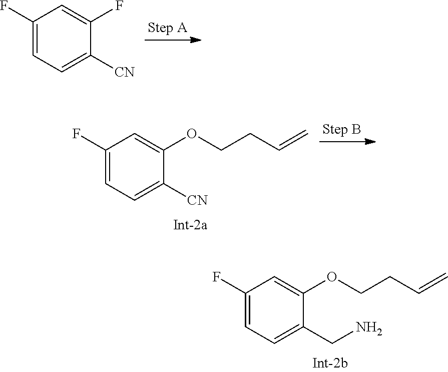

### Step A—Synthesis of Intermediate Compound Int-2a

To a solution of NaH (1.2 g, 30 mmol) in toluene (50 mL) was added but-3-en-1-ol (1.8 g, 25 mmol) dropwise and the mixture was allowed to stir at room temperature for 30 minutes. 2,4-Difluorobenzonitrile (4.2 g, 30 mmol) was then added in one portion and the mixture was allowed to stir at room temperature for 6 hours. Water (30 mL) was slowly added and the mixture was extracted with EtOAc and washed with brine. The organic layer was dried over anhydrous sodium sulfate and concentrated in vacuo. The crude residue was purified using flash column chromatography on silica gel (20:1 PE:EtOAc) to provide compound Int-2b (4.2 g, 78%) as a yellow oil. ¹H NMR (400 MHz, CDCl₃) δ 7.51-7.53 (m, 1H), 7.20-7.25 (m, 1H), 6.79-6.81 (m, 1H), 5.85-5.89 (m, 1H), 5.05-5.11 (m, 2H), 4.05-4.11 (m, 2H), 2.47-2.51 (m, 2H).

### Step B—Synthesis of Intermediate Compound Int-2b

To a solution of compound Int-2a (2.8 g, 15 mmol) in anhydrous THF (60 mL) cooled to 0° C. was added LiAlH₄ (570 mg, 15 mmol) portionwise. The mixture was warmed to room temperature and stirred for about 15 hours. The reaction was quenched by the sequential slow addition of water (1.0 mL), 10% sodium hydroxide solution (2.0 mL) and water (4.0 mL). The resulting mixture was filtered through a pad of celite and washed with THF (20 mL) and the filtrate was concentrated in vacuo to provide compound Int-2b (2.4 g, 82%) as a yellow oil. LCMS anal. calcd. for C₁₁H₁₄FNO: 195.1. Found: 196.1 (M+1)⁺.

### Example 3

### Preparation of Compound Int-3b

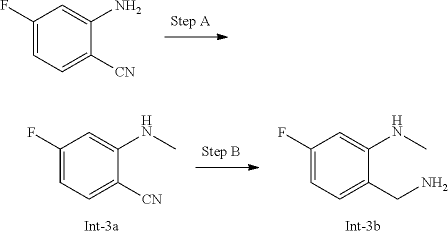

### Step A—Synthesis Intermediate Compound Int-3a

To a solution of 2-amino-4-fluorobenzonitrile (1.0 g, 7.4 mmol) in DMA (10 mL) was added dimethyl oxalate (1.3 g, 11 mmol), followed by KOtBu (1.0 g, 9.0 mmol). The mixture was heated to reflux for 16 hours then cooled to room temperature and concentrated in vacuo. The resulting residue was dissolved in EtOAc (50 mL) and washed with water and brine. The organic layer was concentrated in vacuo and the residue obtained was purified using flash column chromatography on silica gel (4/1 PE/EtOAc) to provide compound Int-3a (600 mg, 58%) as a yellow oil. ¹H NMR (400 MHz, CDCl₃) δ 7.19-7.25 (m, 1H), 6.39-6.47 (m, 2H), 2.95 (s, 3H).

### Step B—Synthesis of Intermediate Compound Int-3b

To a solution of compound Int-3a (540 mg, 3.5 mmol) in anhydrous THF (10 mL) cooled to 0° C. was added LiAlH₄ (380 mg, 10 mmol) portionwise. The mixture was warmed to room temperature and stirred for about 15 hours. The reaction was quenched by the sequential slow addition of water (1.0 mL), 10% sodium hydroxide solution (2.0 mL) and water (4.0 mL). The resulting mixture was filtered through a pad of Celite and washed with THF (20 mL) and the filtrate was concentrated in vacuo to provide compound Int-3b (500 mg, 92%) as a yellow oil. ¹H NMR (400 MHz, CDCl₃) δ 6.91-6.96 (m, 1H), 6.31-6.38 (m, 2H), 6.05 (br, 1H), 3.82 (s, 2H), 2.85 (s, 3H).

### Example 4

### Preparation of Compound Int-4d

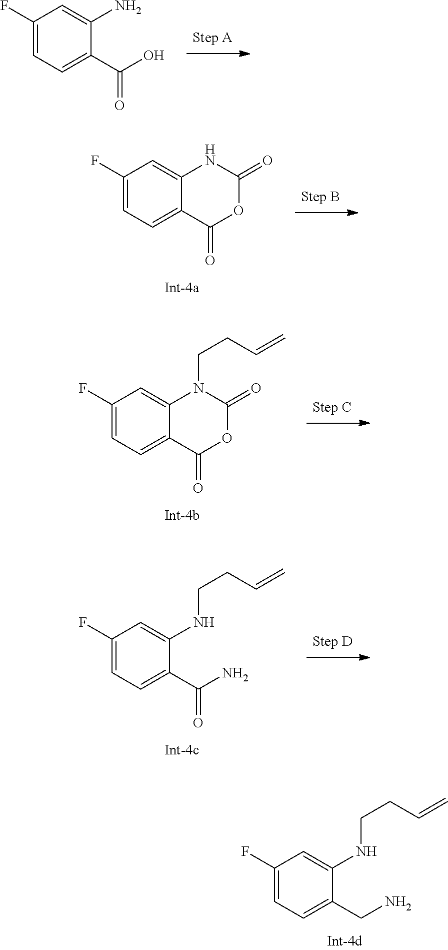 [SMILES: Nc1cc(F)ccc1C(=O)O ; O=c1[nH]c2cc(F)ccc2c(=O)o1 ; C=CCCn1c(=O)oc(=O)c2ccc(F)cc21 ; C=CCCNc1cc(F)ccc1C(N)=O ; C=CCCNc1cc(F)ccc1CN]

### Step A—Synthesis of Intermediate Compound Int-4a

To a solution of 2-amino-4-fluorobenzoic acid (15.5 g, 100 mmol) in THF (200 mL) cooled to 0° C. was added triphosgene (27.9 g, 105 mmol). After heating at 50° C. for 6 hours, the solvent was concentrated in vacuo. The crude residue was triturated with EtOAc/petroleum ether (1:1) and the solid was collected by filtration to provide compound Int-4a (15.7 g, 85%). ¹H NMR (400 MHz, DMSO) δ 11.9 (s, 1H), 7.95-7.98 (m, 1H), 7.06-7.11 (m, 1H), 6.84-6.89 (m, 1H).

### Step B—Synthesis of Intermediate Compound Int-4b

To a mixture of compound Int-4a (14.5 g, 80 mmol), but-3-en-1-ol (11.5 g, 160 mmol), and Ph₃P (31.4 g, 120 mmol) in anhydrous THF (500 mL) cooled to 0° C. was added DIAD (24.1 g, 120 mmol) dropwise. After addition, the mixture was allowed to stir at room temperature for 6 hours. The solvent was concentrated in vacuo and the residue obtained was purified using flash column chromatography on silica gel (2/1 petroleum ether/EtOAc) to provide compound Int-4b (12.4 g, 60%) as a yellow oil. ¹H NMR (400 MHz, CDCl₃) δ 8.16-8.23 (m, 1H), 6.97-7.03 (m, 1H), 6.85-6.91 (m, 1H), 5.83-5.91 (m, 1H), 5.13-5.17 (m, 2H), 4.07-4.11 (t, J=7.6 Hz, 2H), 2.48-2.54 (m, 2H).

### Step C—Synthesis of Intermediate Compound Int-4c

To a solution of compound Int-4b (10 g, 42 mmol) in THF (150 mL) was added 28% NH₃.H₂O (25 mL) and the mixture was allowed to stir at 60° C. for about 15 hours. The solvent was removed in vacuo and the residue obtained was purified at (3/1 petroleum ether/EtOAc) to provide compound Int-4c (6.6 g, 75%) as a white foam.

### Step D—Synthesis of Intermediate Compound Int-4d

To a solution of compound Int-4c (1.2 g, 5.8 mmol) in THF was added LiAlH₄ (1.2 g, 31.5 mmol) at 0° C. After stirring at 80° C. for 3 hours, the reaction was cooled to 0° C. and quenched by the slow addition of water (6 mL) and 2.4 mL of 10% NaOH solution (2.4 mL). The mixture was filtered through a pad of Celite and the filtrate was concentrated in vacuo. The resulting residue was purified using prep-TLC to provide compound Int-4d (260 mg, 46%). ¹H NMR (400 MHz, CDCl₃) δ 6.91-6.96 (m, 1H), 6.31-6.38 (m, 2H), 6.05 (br, 1H), 5.86-5.91 (m, 1H), 5.11-5.15 (m, 2H), 3.82 (s, 2H), 2.85 (m, 2H), 2.41 (m, 2H).

### Example 5

### Preparation of Compound Int-5b

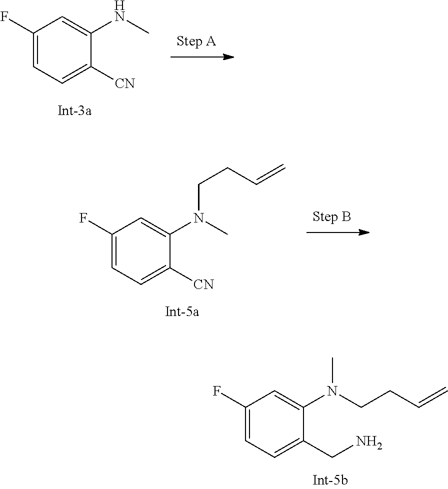

### Step A—Synthesis of Intermediate Compound Int-5a

To a solution of compound Int-3a (450 mg, 3.0 mmol) in anhydrous DMF (20 mL) in a sealed tube cooled to 0° C. was added NaH (240 mg, 6.0 mmol). After stirring at room temperature for 30 min, 4-bromobut-1-ene (810 mg, 6.0 mmol) was added and the mixture was heated to 90° C. in a sealed tube for 24 hours. The solvent was removed in vacuo and the resulting residue was dissolved in EtOAc (50 mL), washed with water and brine. The organic layer was concentrated in vacuo and the residue obtained was purified using prep-TLC (5/1 petroleum ether/EtOAc) to provide compound Int-5a (200 mg, 28%).

### Step B—Synthesis of Intermediate Compound Int-5b

To a solution of compound Int-5a (204 mg, 1.0 mmol) in anhydrous THF (5 mL) cooled to 0° C. was added LiAlH₄ (95 mg, 2.5 mmol) portionwise. The mixture was warmed to room temperature and stirred for about 15 hours. The reaction was quenched by the sequential slow addition of water (1 mL), 10% sodium hydroxide solution (2 mL) and water (4 mL). The resulting mixture was filtered through a pad of Celite and washed with THF (20 mL) and the filtrate was concentrated in vacuo to provide compound Int-5b (180 mg, 90%) as a yellow oil. ¹H NMR (400 MHz, CDCl₃) δ 6.91-6.95 (m, 1H), 6.31-6.37 (m, 2H), 5.97-6.01 (m, 1H), 5.13-5.15 (m, 2H), 3.86 (s, 2H), 2.93-2.97 (m, 5H), 2.41 (m, 2H). LCMS anal. calcd. for C₁₂H₁₇N₂F: 208.1. Found: 208.2 (M+1)⁺.

### Example 6

### Preparation of Compound Int-6b

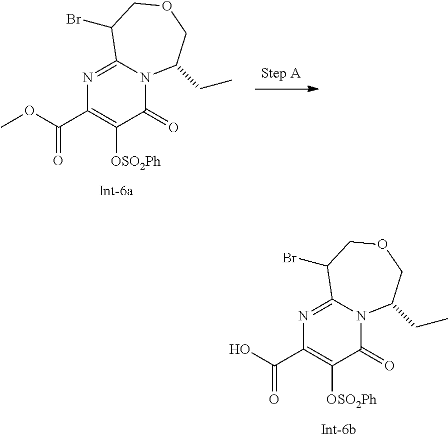

To a solution of compound Int-6a (500 mg, 1.03 mmol) in MeOH/THF (1/1, 10 mL) cooled to 0° C. was slowly added aqueous LiOH (1.0 M, 2.1 mL, 2.1 mmol). After stirring for 1 hour at room temperature, the mixture was neutralized with 1.0 M HCl solution (6.0 mL, 6.0 mmol) and extracted with EtOAc. The organic layer was washed with brine, dried over sodium sulfate concentrated in vacuo to provide compound Int-6b (384 mg, 81%) as an oil. LCMS anal. calcd. for C₁₇H₁₇BrN₂O₇S: 471.9. Found: 472.9 (M+1)⁺.

### Example 7

### Preparation of Compound 1

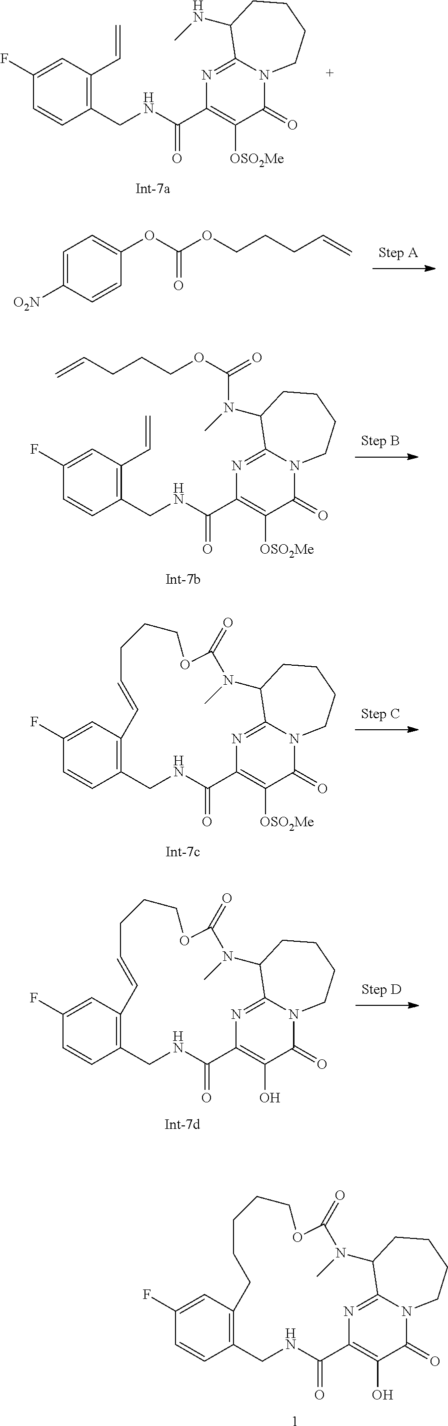

### Step A—Synthesis of Intermediate Compound Int-7b

To a mixture of compound Int-7a (465 mg, 1.0 mmol) and Carbonic acid 4-nitro-phenyl ester pent-4-enyl ester (500 mg, 2.0 mmol) in dichloromethane (20 mL) was added TEA (200 mg, 2.0 mmol) and the mixture was allowed to stir at room temperature for 16 hours. The mixture was concentrated and the resulting residue was purified using prep-TLC (4/1 petroleum ether/EtOAc) to provide compound Int-7b (90 mg, 15%). LCMS anal. calcd. for C₂₇H₃₃FN₄O₇S: 576.2. Found: 577.1 (M+1)⁺.

### Step B—Synthesis of Intermediate Compound Int-7c

A solution of compound Int-7b (300 mg, 0.51 mmol) in dichloromethane (120 mL) was degassed with nitrogen for 30 minutes. Zhan-1B Catalyst (30 mg, 0.04 mmol) was then added and the mixture was allowed to stir at room temperature for about 15 hours under a nitrogen atmosphere. The mixture was concentrated in vacuo and the resulting residue was purified using prep-TLC (4/1 petroleum ether/EtOAc) to provide compound Int-7c (280 mg, 97%). LCMS anal. calcd. for C₂₅H₂₉FN₄O₇S: 548.1. Found: 549.2 (M+1)⁺.

### Step C—Synthesis of Intermediate Compound Int-7d

To a solution of compound Int-7c (100 mg, 0.20 mmol) in THF/H₂O (3 mL, 2/1) was added LiOH monohydrate (12 mg, 0.3 mmol). The mixture was allowed to stir at room temperature for 30 minutes and diluted with dichloromethane (10 mL). The organic layer was separated, dried over Na₂SO₄, filtered and concentrated in vacuo to provide compound Int-7d (20 mg, 82%). LCMS anal. calcd. for C₂₄H₂₇FN₄O₅: 470.2. Found: 471.2 (M+1)⁺.

### Step D—Synthesis of Compound 1

To a solution of compound Int-7d (95 mg, 0.2 mmol) in EtOAc (20 mL) was added 10% Pd/C (50 mg). The mixture was allowed to stir under an atmosphere of H₂ under balloon pressure for 12 hours. The mixture was filtered through Celite, rinsed with EtOAc, and the filtrate was concentrated in vacuo. The resulting residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 40-70%, 0-12 min] to provide Compound 1 (40 mg, 42%). ¹H NMR (400 MHz, CDCl₃) δ 11.96 (s, 1H), 7.36-7.37 (m, 1H), 6.88-6.92 (m, 2H), 5.20-5.32 (m, 2H), 4.42-4.65 (m, 2H), 4.20-4.32 (m, 1H), 3.82-3.92 (m, 1H), 3.36-3.44 (m, 1H), 2.82-2.95 (m, 1H), 2.78 (s, 3H), 2.60-2.70 (m, 1H), 2.02-2.18 (m, 2H), 1.70-1.85 (m, 2H), 1.50-1.75 (m, 6H), 1.30-1.50 (m, 3H). LCMS anal. calcd. for C₂₄H₂₉FN₄O₅: 472.2. Found: 473.1 (M+1)⁺.

### Example 8

### Preparation of Compound 2

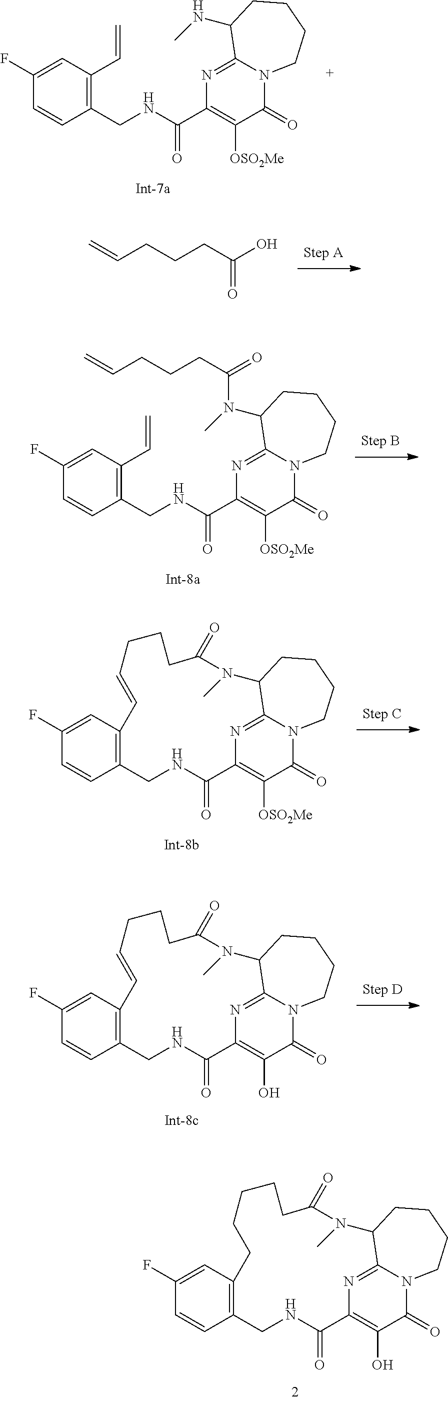

### Step A—Synthesis of Intermediate Compound Int-8a

To a solution of compound Int-7a (100 mg, 0.215 mmol), hex-5-enoic acid (74 mg, 0.646 mmol), and HATU (204 mg, 0.538 mmol) in DMF (10 mL) was added DIPEA (40 mg, 1.05 mmol) and the mixture was allowed to stir at room temperature for about 15 hours. The mixture was concentrated and purified using prep-TLC (1/3 petroleum ether/EtOAc) to provide compound Int-8a (80 mg, 67%). ¹H NMR (400 MHz, MeOD) δ 7.26-7.47 (m, 2H), 7.20-7.26 (m, 2H), 7.02-7.18 (m, 1H), 6.48 (s, 1H), 5.68-5.70 (m, 2H), 5.43-5.45 (m, 2H), 4.68 (s, 3H), 3.95-3.97 (m, 3H), 3.43 (s, 4H), 3.23 (s, 4H), 2.60-2.63 (m, 3H), 1.86-1.92 (m, 6H). LCMS anal. calcd. For C₂₇H₃₃FN₄O₆S: 560.2. Found: 561.1 (M+1)⁺.

### Step B—Synthesis of Intermediate Compound Int-8b

To a solution of compound Int-8a (50 mg, 0.09 mmol) in dichloromethane (100 mL) was added Zhan-1b Catalyst (9 mg, 0.01 mmol) and the mixture was allowed to stir at room temperature for 24 hours. The mixture was concentrated in vacuo (bath temp<25° C.) and the resulting residue was purified using prep-TLC (1/3 petroleum ether/EtOAc) to provide compound Int-8b (40 mg, 85%). ¹H NMR (400 MHz, CDCl₃) δ 7.29-7.36 (m, 3H), 6.89-6.90 (m, 1H), 6.54-6.58 (m, 1H), 6.23-6.27 (m, 1H), 5.86-5.88 (m, 1H), 5.16-5.21 (m, 1H), 4.50-4.61 (m, 2H), 3.55 (s, 3H), 2.94 (s, 3H), 2.68 (s, 3H), 2.34-2.38 (m, 1H), 2.23-2.26 (m, 1H), 2.06-2.17 (m, 4H), 1.79-1.80 (m, 2H), 1.76-1.78 (m, 1H). LCMS anal. calcd. for C₂₅H₂₉FN₄O₆S: 532.1. Found: 533.1 (M+1)⁺.

### Step C—Synthesis of Intermediate Compound Int-8c

To a solution of compound Int-8b (100 mg, 0.19 mmol) in THF (5.0 mL) was added LiOH monohydrate (42 mg, 1.0 mmol) and the mixture was allowed to stir at room temperature for 1 hour. Water was then added and the mixture was extracted with dichloromethane. The combined organic layer was dried over Na₂SO₄, filtered and concentrated in vacuo to provide compound Int-8c (75 mg, 88%) which was used directly in the next step. LCMS anal. calcd. for C₂₄H₂₇FN₄O₆S: 454.2. Found: 455.2 (M+1)⁺.

### Step D—Synthesis of Compound 2

A mixture of compound Int-8c (50 mg, 0.11 mmol) and 10% Pd/C (10 mg) in CH₃OH was allowed to stir at room temperature for about 15 hours under an atmosphere of H₂. The mixture was filtered and concentrated. The resulting residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 35 mL/min, Gradient: 44-64%, 0-12 min] to provide Compound 2 (20 mg, 40%). ¹H NMR (400 MHz, CDCl₃) δ 7.27-7.29 (m, 1H), 7.01-7.06 (m, 2H), 6.86-6.92 (m, 1H), 5.76 (d, J=10.4 Hz, 1H), 4.66-4.69 (m, 1H), 4.22-4.25 (m, 1H), 3.48-3.55 (m, 1H), 2.80 (s, 3H), 2.61-2.62 (m, 1H), 2.50-2.53 (m, 2H), 2.16-2.30 (m, 1H), 2.02-2.04 (m, 1H), 1.80-1.81 (m, 2H), 1.68-1.79 (m, 5H), 1.41-1.52 (m, 3H), 1.32 (s, 1H) LCMS anal. calcd. for C₂₄H₂₉FN₄O₆S: 456.1. Found: 457.1 (M+1)⁺.

### Example 9

### Preparation of Compound 3

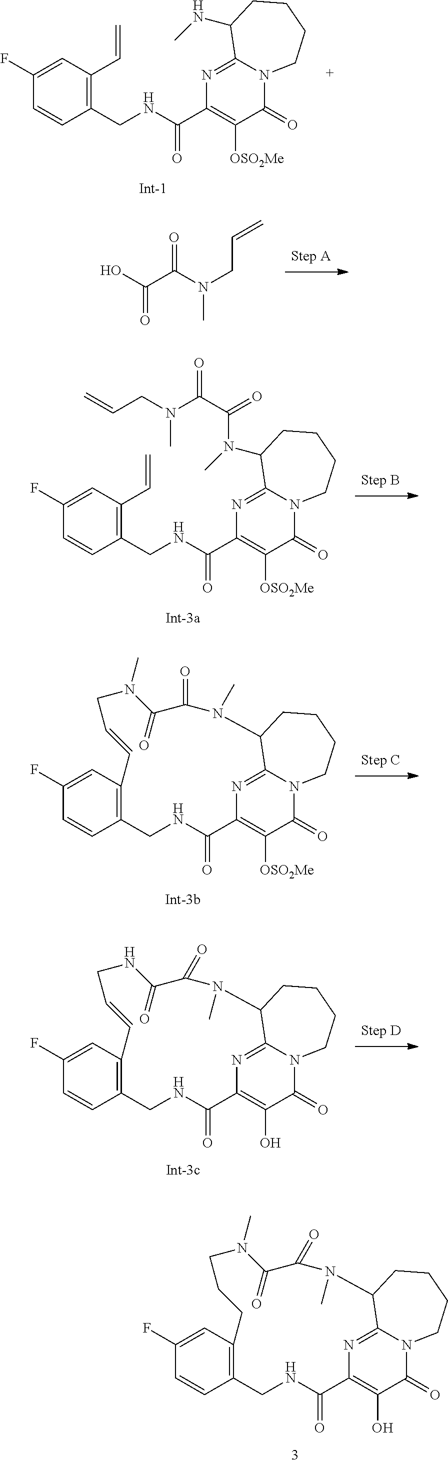

### Step A—Synthesis of Intermediate Compound Int-9a

To a solution of compound Int-7a (372 mg, 0.8 mmol), DIPEA (515 mg, 4 mmol) and N-Allyl-N-methyl-oxalamic acid (258 mg, 2 mmol) in DMF (5 mL) was added PyClu (540 mg, 1.5 mmol) and the mixture was allowed to stir at room temperature for 1 hour. The reaction was diluted with EtOAc (50 mL), washed with water and brine, and the organic layer was dried over Na₂SO₄. The solvent was removed in vacuo and the resulting residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 15 mL/min, Gradient: 35-65%, 0-12 min] to provide compound Int-9a (300 mg, 63.6%). LCMS anal. calcd. for C₂₇H₃₂FN₅O₇S: 589.2. Found: 590.1 (M+1)⁺.

### Step B—Synthesis of Intermediate Compound Int-9b

A solution of compound Int-9a (150 mg, 0.26 mmol) in dichloromethane (100 mL) was degassed with nitrogen for 30 minutes. Zhan-1b Catalyst (16.2 mg, 0.02 mmol) was then added and the mixture was allowed to stir at room temperature for 60 hours under an atmosphere of N₂. The mixture was concentrated in vacuo (bath temp<25° C.) and the residue obtained was purified using prep-TLC (20/1 dichloromethane/CH₃OH) to provide compound Int-9b (100 mg, 67%). ¹H NMR (400 MHz, CDCl₃) δ 7.39-7.42 (m, 1H), 7.31-7.33 (m, 1H), 6.95-6.98 (m, 2H), 6.85-6.88 (m, 1H), 6.75-6.80 (m, 1H), 4.67-4.74 (m, 1H), 4.45-4.46 (m, 1H), 3.45-3.47 (m, 1H), 3.30-3.33 (m, 1H), 3.16-3.17 (m, 1H), 3.04 (s, 6H), 2.94 (s, 3H), 2.71-2.74 (m, 1H), 2.06-2.11 (m, 5H), 1.83-1.88 (m, 1H), 1.50-1.52 (m, 1H). LCMS anal. calcd. for C₂₅H₂₈FN₅O₇S: 561.1. Found: 562.1 (M+1)⁺.

### Step C—Synthesis of Intermediate Compound Int-9c

To a solution of compound Int-9b (100 mg, 0.18 mmol) in THF (5 mL) was added LiOH monohydrate (42 mg, 1.0 mmol) and the mixture was allowed to stir at room temperature for 1 hour. Water was added and the reaction was extracted with EtOAc. The combined organic layer was dried over Na₂SO₄, filtered and concentrated in vacuo to provide compound Int-9c (70 mg, 78%). LCMS anal. calcd. for C₂₄H₂₆FN₅O₅: 483.2. Found: 484.2 (M+1)⁺.

### Step D—Synthesis of Compound 3

A mixture of compound Int-9c (70 mg, 0.17 mmol) and 10% Pd/C (10 mg) in EtOAc (20 mL) was allowed to stir at room temperature for about 15 hours under an atmosphere of H₂. The mixture was filtered, concentrated in vacuo, and the residue obtained was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 17 mL/min, Gradient: 39-59%, 0-12 min] to provide Compound 3 (15 mg, 24%). ¹H NMR (400 MHz, CDCl₃) δ 7.39-7.42 (m, 1H), 7.31-7.33 (m, 1H), 6.95-6.98 (m, 2H), 5.55-5.58 (m, 1H), 5.27-5.30 (m, 1H), 4.67-4.74 (m, 1H), 4.45-4.46 (m, 1H), 3.45-3.47 (m, 1H), 3.30-3.33 (m, 1H), 3.16-3.17 (m, 1H), 3.04 (s, 6H), 2.71-2.74 (m, 1H), 2.06-2.11 (m, 5H), 1.83-1.88 (m, 3H), 1.50-1.52 (m, 1H). LCMS anal. calcd. for C₂₄H₂₈FN₅O₅S: 485.2. Found: 486.1 (M+1)⁺.

### Example 10

### Preparation of Compound 4

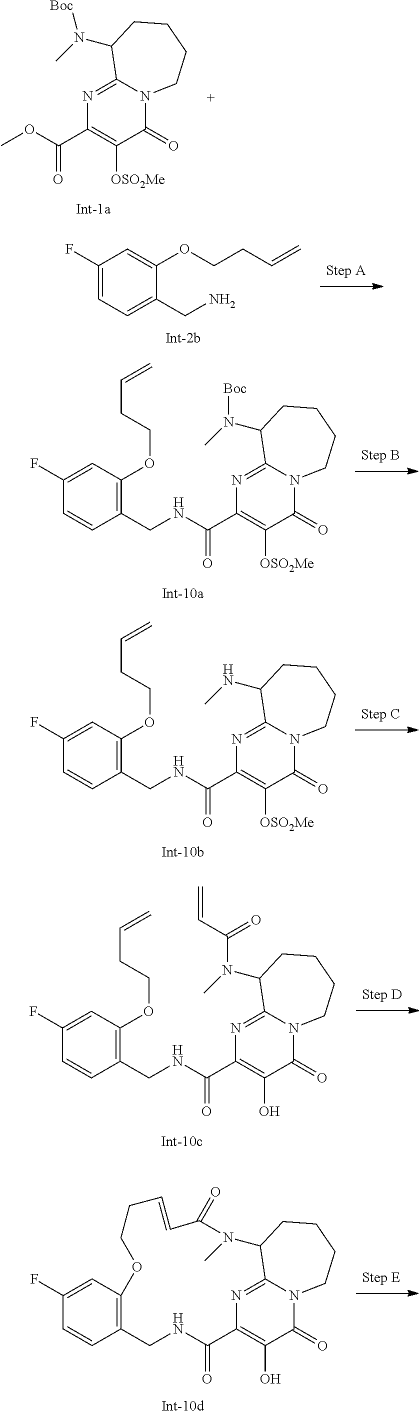

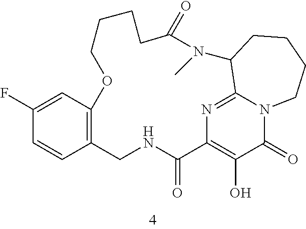 [SMILES: CN1C(=O)CCCCOc2cc(F)ccc2CNC(=O)c2nc3n(c(=O)c2O)CCCCC31]

### Step A—Synthesis of Intermediate Compound Int-10a

To a solution of compound Int-2b (310 mg, 1.6 mmol) in dichloromethane (5.0 mL) cooled to 0° C. was added trimethylaluminum (2.0 M solution in toluene, 1.0 mL, 2.0 mmol) dropwise. After stirring for 30 minutes at 0° C., Int-1a (500 mg, 1.1 mmol) in dichloromethane (1.0 mL) was added dropwise. After addition, the mixture was allowed to stir at 40° C. for about 15 hours. The mixture was cooled to room temperature, poured into an aqueous HCl solution (1.0 M, 10.0 mL), and extracted with dichloromethane (3×30 mL). The combined organic layer was washed with brine and dried over anhydrous sodium sulfate. The solvent was removed in vacuo and the residue obtained was purified using prep-TLC (30/1 dichloromethane/MeOH) to provide compound Int-10a (300 mg, 51%) as a yellow oil. ¹H NMR (400 MHz, CDCl₃) δ 7.19-7.23 (m, 1H), 6.58-6.62 (m, 2H), 5.85-5.91 (m, 2H), 5.13-5.21 (m, 3H), 4.61-4.64 (m, 1H), 4.43-4.49 (m, 1H), 4.01-4.08 (m, 2H), 3.48-3.52 (m, 1H), 3.07 (s, 3H), 2.51-2.54 (m, 2H), 1.97-2.03 (m, 4H), 1.53-1.58 (m, 6H), 1.47-1.51 (m, 9H). LCMS anal. calcd. for C₂₈H₃₇FN₄O₈S: 608.2. Found: 609.1 (M+1)⁺.

### Step B—Synthesis of Intermediate Compound Int-10b

To a solution of compound Int-10a (300 mg, 0.50 mmol) in dichloromethane (3.0 mL) cooled to 0° C. was added TFA (1.0 mL). After addition, the mixture was allowed to stir at room temperature for 3 hours. The solvent was removed in vacuo to provide compound Int-10b (255 mg, 99%) as a yellow oil. LCMS anal. calcd. for C₂₃H₂₉FN₄O₆S: 508.1. Found: 509.1 (M+1)⁺.

### Step C—Synthesis of Intermediate Compound Int-10c

To a solution of compound Int-10b (230 mg, 0.45 mmol) and triethylamine (101 mg, 1.0 mmol) in dichloromethane (5.0 mL) was slowly added acryloyl chloride (45 mg, 0.50 mmol) in dichloromethane (1.0 mL). After addition, the mixture was allowed to stir at room temperature for 6 hours then diluted with dichloromethane (30 mL). The mixture was washed with 1.0 M HCl solution, brine and dried over anhydrous sodium sulfate. The solvent was removed in vacuo and the residue obtained was purified using prep-TLC (20/1 dichloromethane/MeOH) to provide compound Int-10c (184 mg, 80%) as a yellow solid. ¹H NMR (400 MHz, CDCl₃) δ 9.63 (br, 1H), 7.23-7.25 (m, 1H), 6.57-6.62 (m, 3H), 5.85-5.91 (m, 4H), 5.11-5.21 (m, 3H), 4.62-4.65 (m, 1H), 4.47-4.51 (m, 1H), 4.05-4.12 (m, 2H), 3.48-3.52 (m, 2H), 3.08 (s, 3H), 2.51-2.54 (m, 2H), 1.97-2.03 (m, 4H), 1.47-1.62 (m, 2H). LCMS anal. calcd.- or C₂₅H₂₉FN₄O₅: 484.2. Found: 485.1 (M+1)⁺.

### Step D—Synthesis of Intermediate Compound Int-10d

A solution of compound Int-10c (180 mg, 0.36 mmol) in dichloromethane (70 mL) was degassed with nitrogen for 30 minutes. Zhan-1b catalyst (30 mg, 0.042 mmol) was added and the mixture was allowed to stir at room temperature under a nitrogen atmosphere for about 15 hours. The solvent was removed in vacuo and the residue obtained was purified using prep-TLC (15/1 dichloromethane/MeOH) to provide compound Int-10d (100 mg, yield: 51%) as a yellow solid. LCMS anal. calcd. for C₂₃H₂₅FN₄O₅: 456.2. Found: 457.1 (M+1)⁺.

### Step E—Synthesis of Compound 4

A solution of compound Int-10d (100 mg, 0.21 mmol) in EtOAc (10 mL) was purged with nitrogen and 10% Pd/C (30 mg) was added. The mixture was allowed to stir over hydrogen atmosphere under balloon pressure for 2 hours. The mixture was filtered through Celite, washed with EtOAc (10 mL), and the solvent was removed in vacuo. The crude residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 43-63%, 0-12 min] to provide Compound 4 (20 mg, 21%) as a white solid. ¹H NMR (400 MHz, CDCl₃) δ 12.07 (br, 1H), 7.83-7.85 (br, 1H), 7.29-7.32 (m, 1H), 6.61-6.71 (m, 2H), 5.87-5.89 (d, J=10.8 Hz, 1H), 5.22-5.27 (m, 1H), 4.45-4.54 (m, 1H), 4.40-4.51 (m, 1H), 4.10-4.14 (m, 1H), 3.47-3.51 (m, 2H), 3.07 (s, 3H), 2.71-2.74 (m, 1H), 2.31-2.35 (m, 1H), 1.97-2.03 (m, 8H), 1.71-1.79 (m, 1H), 1.43-1.49 (m, 1H). LCMS anal. calcd. for C₂₃H₂₇FN₄O₅: 458.1. Found: 459.1 (M+1)⁺.

### Example 11

### Preparation of Compound 5

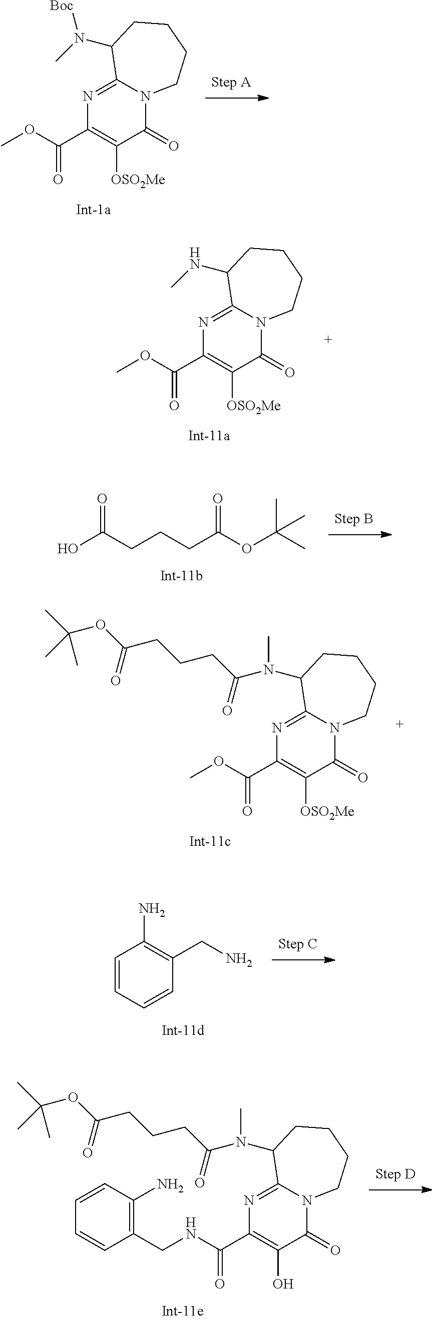

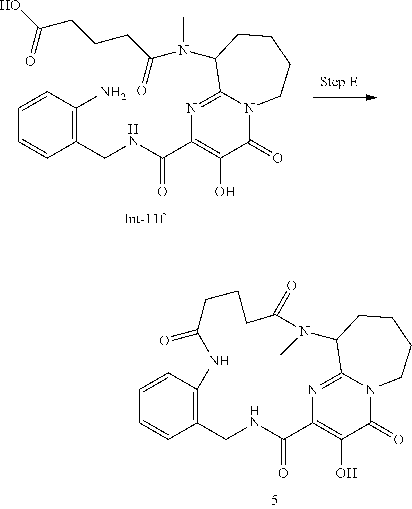 [SMILES: CN(C(=O)CCCC(=O)O)C1CCCCn2c1nc(C(=O)NCc1ccccc1N)c(O)c2=O ; CN1C(=O)CCCC(=O)Nc2ccccc2CNC(=O)c2nc3n(c(=O)c2O)CCCCC31]

### Step A—Synthesis of Intermediate Compound Int-11a

To a solution of compound Int-1a (500 mg, 1.1 mmol) in dichloromethane (6.0 mL) was added TFA (2.0 ml) and the mixture was allowed to stir at room temperature for 3 hours. The solvent was removed in vacuo to provide compound Int-11a (380 mg, 99%). LCMS anal. calcd. for C₁₃H₁₉N₃O₆S: 345.1. Found: 346.1 (M+1)⁺.

### Step B—Synthesis of Intermediate Compound Int-11c

To a solution of compound Int-11a (380 mg, 1.1 mmol) in DMF (10.0 mL) was added 5-tert-butoxy-5-oxopentanoic acid (Int-11b) (210 mg, 1.1 mmol), DIPEA (200 mg, 1.6 mmol) and PyClu (480 mg, 1.5 mmol). After stirring at room temperature for about 15 hours, the mixture was concentrated in vacuo and the resulting residue was dissolved in dichloromethane (50.0 mL). The solution was washed with a 1.0 M HCl solution and brine and then dried over anhydrous sodium sulfate. The solvent was removed in vacuo and the residue obtained was purified using prep-TLC (50/1 dichloromethane/MeOH) to provide compound Int-11c (310 mg, 53%). ¹H NMR (400 MHz, CDCl₃) δ 5.83-5.85 (d, J=8.0 Hz, 1H), 5.18-5.23 (m, 1H), 3.95 (s, 3H), 3.54-3.60 (m, 1H), 3.51 (s, 3H), 3.12 (s, 3H), 2.44-2.48 (m, 2H), 2.28-2.32 (m, 2H), 2.08-2.13 (m, 2H), 1.83-1.95 (m, 4H), 1.49 (s, 9H), 1.12-1.21 (m, 2H). LCMS anal. calcd. for C₂₂H₃₃N₃O₉S: 515.1. Found: 538.1 (M+Na)⁺.

### Step C—Synthesis of Intermediate Compound Int-11e

A mixture of compound Int-11c (260 mg, 0.50 mmol) and 2-(aminomethyl) aniline (Int-11d) (180 mg, 1.5 mmol) in EtOH (3.0 mL) was heated to 120° C. under microwave radiation for 2 hours. The solvent was removed in vacuo and the residue obtained was purified using prep-TLC (20/1 dichloromethane/MeOH) to provide compound Int-11e (143 mg, 51%) as a yellow oil. ¹H NMR (400 MHz, CDCl₃) δ 7.29-7.37 (m, 4H), 5.83-5.85 (d, J=8.0 Hz, 1H), 5.18-5.23 (m, 1H), 4.71-4.75 (m, 1H), 4.51-4.61 (m, 1H), 3.54-3.60 (m, 1H), 3.12 (s, 3H), 2.47-2.51 (m, 2H), 2.29-2.33 (m, 2H), 2.11-2.15 (m, 2H), 1.81-1.95 (m, 4H), 1.51 (s, 9H), 1.15-1.21 (m, 2H). LCMS anal. calcd. for C₂₇H₃₇N₅O₆: 527.2. Found: 528.1 (M+1)⁺.

### Step D—Synthesis of Intermediate Compound Int-11f

To a solution of compound Int-11e (100 mg, 0.20 mmol) in dichloromethane (4.0 mL) was added TFA (1.0 mL) and the mixture was allowed to stir at room temperature for 4 hours. The solvent was removed in vacuo to provide compound Int-11f (91 mg, 100%) as a yellow oil. LCMS anal. calcd. for C₂₃H₂₉N₅O₆: 471.2. Found: 472.1 (M+1)⁺.

### Step E—Synthesis of Compound 5

To a solution of compound Int-11f (60 mg, 0.13 mmol) in DMF (15 mL) cooled to 0° C. was added DIEA (51 mg, 0.40 mmol) and PyClu (63 mg, 0.20 mmol). After stirring at room temperature for about 15 hours, the solvent was removed in vacuo and the residue obtained was purified using prep-HPLC [Column-type: Boston symmetrix C18ODS-R (150 mm×30 mm, 5 μm). Mobile phase A: water (containing 0.075% TFA, V/V), Mobile phase B: acetonitrile, Flow rate: 40 mL/min, Gradient: 10-40%, 0-12 min] to provide Compound 5 (12.5 mg, 23%) as pink solid. ¹H NMR (400 MHz, CDCl₃) δ 7.62 (br, 1H), 7.47-7.51 (m, 1H), 7.3-7.35 (m, 3H), 7.22 (br, 1H), 5.83-5.85 (d, J=8.0 Hz, 1H), 5.21-5.23 (m, 1H), 4.74-4.82 (m, 1H), 4.54-4.62 (m, 1H), 3.48-3.52 (m, 1H), 3.09 (s, 3H), 2.53-2.56 (m, 2H), 2.46-2.51 (m, 2H), 2.21-2.42 (m, 6H), 1.71-1.75 (m, 1H), 1.31-1.37 (m, 1H). LCMS anal. calcd. for C₂₃H₂₇N₅O₄: 453.2. Found: 454.1 (M+1)⁺.

### Example 12

### Preparation of Compound 6

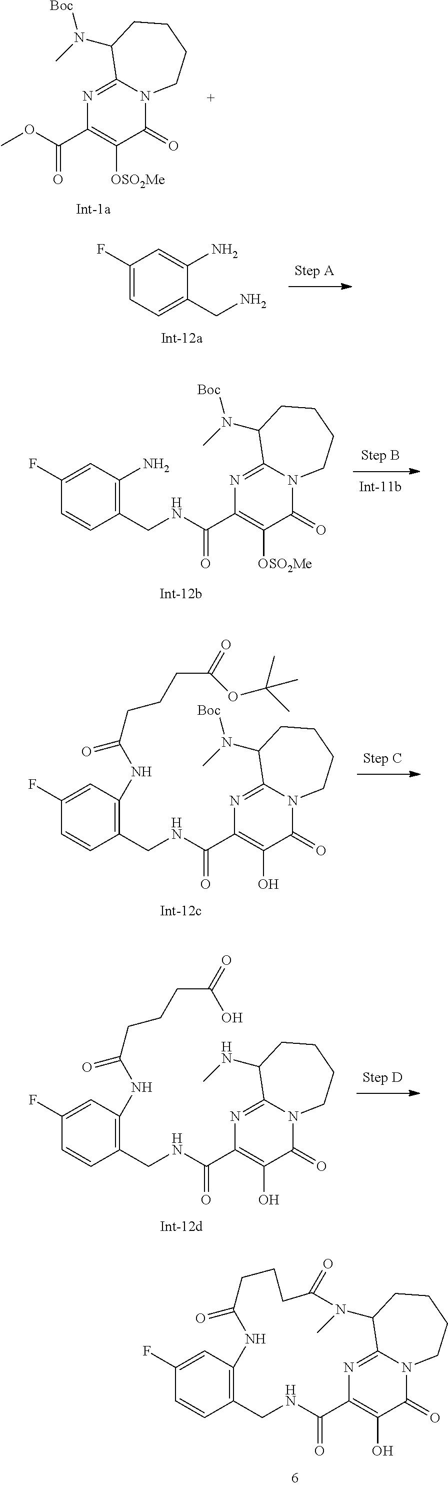

### Step A—Synthesis of Intermediate Compound Int-12b

A mixture of compound Int-1a (445 mg, 1.0 mmol) and 2-aminomethyl-5-fluoro-aniline Int-12a (560 mg, 4.0 mmol) in EtOH (2.5 mL) was heated to 120° C. for 2 hours in a microwave. The mixture was purified using prep-TLC (15/1 dichloromethane/MeOH) to provide compound Int-12b (400 mg, 75%). LCMS anal. calcd. for: C₂₃H₃₀FN₅O₅ 475.2. Found: 476.1 (M+1)⁺.

### Step B—Synthesis of Intermediate Compound Int-6c

To a solution of compound Int-12b (400 mg, 0.70 mmol) and Pentanedioic acid mono-tert-butyl ester Int-10b (263 mg, 1.40 mmol) in dichloromethane (5 mL) was added HATU (420 mg, 1.40 mmol) and DIEA (180 mg, 1.40 mmol). The mixture was allowed to stir for 3 hours at 38° C. and then quenched by addition of aqueous NH₄Cl. The mixture was extracted with dichloromethane and the combined organic layer was dried over Na₂SO₄, filtered and concentrated in vacuo. The crude residue was purified using prep-TLC to provide the product Int-12c (400 mg, 93%). LCMS anal. calcd. for: C₃₂H₄₄FN₅O₈ 645.1. Found: 646.2 (M+1)⁺.

### Step C—Synthesis of Intermediate Compound Int-12d

To a solution of compound Int-12c (200 mg, 0.31 mmol) in dichloromethane (5 mL) was added TFA (1 mL). The mixture was allowed to stir for 3 hours at room temperature and then concentrated in vacuo to provide the product Int-12d (100 mg, 66%). LCMS anal. calcd. for: C₂₃H₂₈FN₅O₆ 489.1. Found: 490.2 (M+1)⁺.

### Step D—Synthesis of Compound 6

A mixture of compound Int-12d (100 mg, 0.25 mmol), PyClu (70 mg, 0.50 mmol), and DIPEA (65 mg, 0.50 mmol) in DMF (2 mL) was allowed to stir for 2 hours at room temperature. The mixture was purified directly by prep-HPLC [Column-type: YMC-pack ODS-AQ (150 mm×30 mm, 5 μm). Mobile phase A: water (containing 0.075% TFA, VAT), Mobile phase B: acetonitrile, Flow rate: 25 mL/min, Gradient: 40-70%, 0-12 min] to provide Compound 6 (20 mg, 20%). ¹H NMR (400 MHz, CDCl₃) δ 7.52-7.62 (m, 1H), 7.41-7.51 (m, 1H), 7.26-7.34 (m, 1H), 6.82-6.92 (m, 1H), 5.71 (d, J=12.0 Hz, 1H), 5.20-5.30 (m, 1H), 4.65-4.75 (m, 1H), 4.42-4.55 (m, 1H), 3.40-3.55 (m, 1H), 3.04 (s, 3H), 2.77-2.83 (m, 1H), 2.55-2.62 (m, 2H), 2.32-2.48 (m, 2H), 1.95-2.20 (m, 6H), 1.75-1.88 (m, 1H), 1.32-1.45 (m, 1H). LCMS anal. calcd. for C₂₃H₂₆FN₅O₅: 471.1. Found: 472.1 (M+1)⁺.

### Example 13

### Preparation of Compound 7

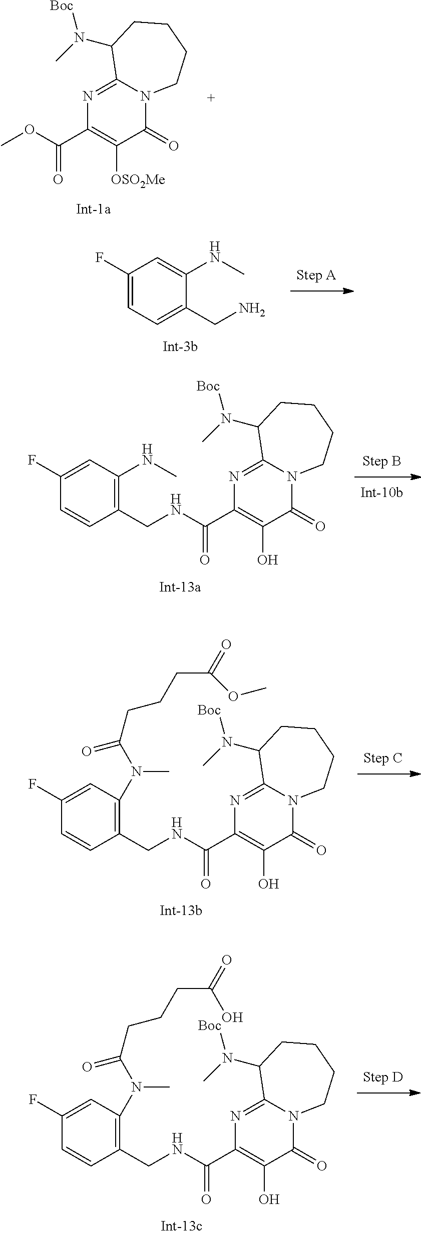

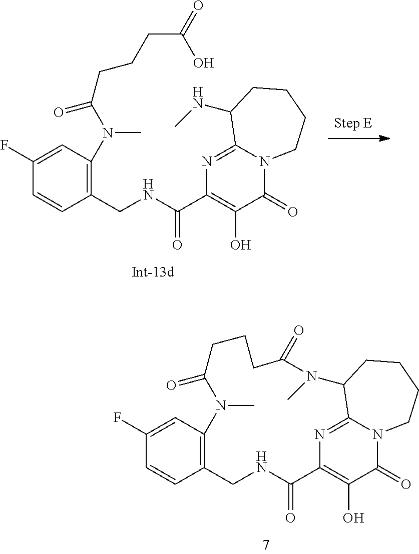 [SMILES: CNC1CCCCn2c1nc(C(=O)NCc1ccc(F)cc1N(C)C(=O)CCCC(=O)O)c(O)c2=O ; CN1C(=O)CCCC(=O)N(C)C2CCCCn3c2nc(c(O)c3=O)C(=O)NCc2ccc(F)cc21]

### Step A—Synthesis of Intermediate Compound Int-13a

A mixture of compound Int-1a (150 mg, 0.34 mmol) and Int-3b (154 mg, 1.0 mmol) in EtOH (3.0 mL) was heated to 120° C. for 2 hours in a microwave. The mixture was cooled to room temperature and the solvent was removed in vacuo. The crude residue was purified using prep-TLC (20/1 dichloromethane/MeOH) to provide compound Int-13a (101 mg, 53%) as a yellow foam. ¹H NMR (400 MHz, CDCl₃) δ 7.78 (s, 1H), 7.41-7.46 (m, 1H), 6.81-6.87 (m, 2H), 5.85-5.91 (m, 1H), 5.21-5.25 (m, 1H), 4.71-4.76 (m, 1H), 4.43-4.49 (m, 1H), 3.51-3.58 (m, 1H), 3.21 (s, 3H), 2.97 (s, 3H), 2.23-2.28 (m, 2H), 1.87-1.93 (m, 4H), 1.53 (s, 9H). LCMS anal. calcd. for C₂₄H₃₂FN₅O₅: 489.2. Found: 490.1 (M+1)⁺.

### Step B—Synthesis of Intermediate Compound Int-13b

To a solution of compound Int-13a (86 mg, 0.15 mmol) in pyridine (3.0 mL) cooled to 0° C. was slowly added methyl 5-chloro-5-oxopentanoate (33 mg, 0.20 mmol) in dichloromethane (1.0 mL). After addition, the mixture was allowed to stir at room temperature for 12 hours then diluted with dichloromethane (50 mL). The mixture was washed with 1.0 M HCl solution, brine and dried over sodium sulfate. The solvent was removed in vacuo and the residue obtained was purified using prep-TLC (20/1 dichloromethane/MeOH) to provide compound Int-13b (40 mg, 38%) as a yellow oil. ¹H NMR (400 MHz, CDCl₃) δ 7.81 (s, 1H), 7.40-7.45 (m, 1H), 6.83-6.88 (m, 2H), 5.87-5.91 (m, 1H), 5.21-5.25 (m, 1H), 4.71-4.76 (m, 1H), 4.43-4.49 (m, 1H), 3.51-3.58 (m, 1H), 3.49 (s, 3H), 3.21 (s, 3H), 2.97 (s, 3H), 2.23-2.27 (m, 4H), 1.81-1.83 (m, 4H), 1.57 (s, 9H), 1.25-1.31 (m, 2H), 1.01-1.05 (m, 2H). LCMS anal. calcd. for C₃₀H₄₀FN₅O₈: 617.2. Found: 618.1 (M+1)⁺.

### Step C—Synthesis of Intermediate Compound Int-13c

To a solution of compound Int-13b (40 mg, 0.066 mmol) in THF (3.0 mL) was added 1.0 M LiOH solution (2.0 mL) and the mixture was allowed to stir at room temperature for 12 hours. The solvent was removed in vacuo, EtOAc (50 mL) was added, and the solution was washed with 1.0 M HCl solution and brine. The organic layer was dried over sodium sulfate, filtered, and concentrated in vacuo to provide compound Int-13c (36 mg, 92%). LCMS anal. calcd. for C₂₉H₃₈FN₅O₈: 603.2. Found: 604.1 (M+1)⁺.

### Step D—Synthesis of Intermediate Compound Int-13d

To a solution of compound Int-13c (36 mg, 0.060 mmol) in dichloromethane (4.0 mL) cooled to 0° C. was added TFA (1.0 mL). After addition, the mixture was allowed to stir at room temperature for 3 hours. The solvent was removed in vacuo to provide compound Int-13d (30 mg, 99%) as a yellow oil. LCMS anal. calcd. for C₂₄H₃₀FN₅O₆: 503.2. Found: 504.1 (M+1)⁺.

### Step E—Synthesis of Compound 7

A mixture of compound Int-13d (30 mg, 0.060 mmol), HOBt (13.8 mg, 0.10 mmol), and DIPEA (51.0 mg, 0.40 mmol) in DMF (10.0 mL) was cooled to 0° C. HATU (38 mg, 0.10 mmol) was added and the mixture was allowed to stir at room temperature for 12 hours. The solvent was removed in vacuo and the residue obtained was purified using prep-HPLC to provide Compound 7 (15.0 mg, 51%) as a white solid. ¹H NMR (400 MHz, CDCl₃) δ 7.71-7.76 (m, 1H), 7.51-7.55 (m, 1H), 7.05-7.11 (m, 1H), 6.83-6.87 (m, 1H), 5.58-5.61 (d, J=10.4 Hz, 1H), 5.25-5.30 (m, 1H), 4.93-4.98 (m, 1H), 3.91-3.96 (m, 1H), 3.47-3.51 (m, 1H), 3.31 (s, 3H), 3.15 (s, 3H), 2.41-2.47 (m, 2H), 1.99-2.21 (m, 6H), 1.71-1.90 (m, 3H), 1.41-1.45 (m, 1H). LCMS anal. calcd. for C₂₄H₂₈FN₅O₅: 485.2. Found: 486.1 (M+1)⁺.

### Example 14

### Preparation of Compound 8

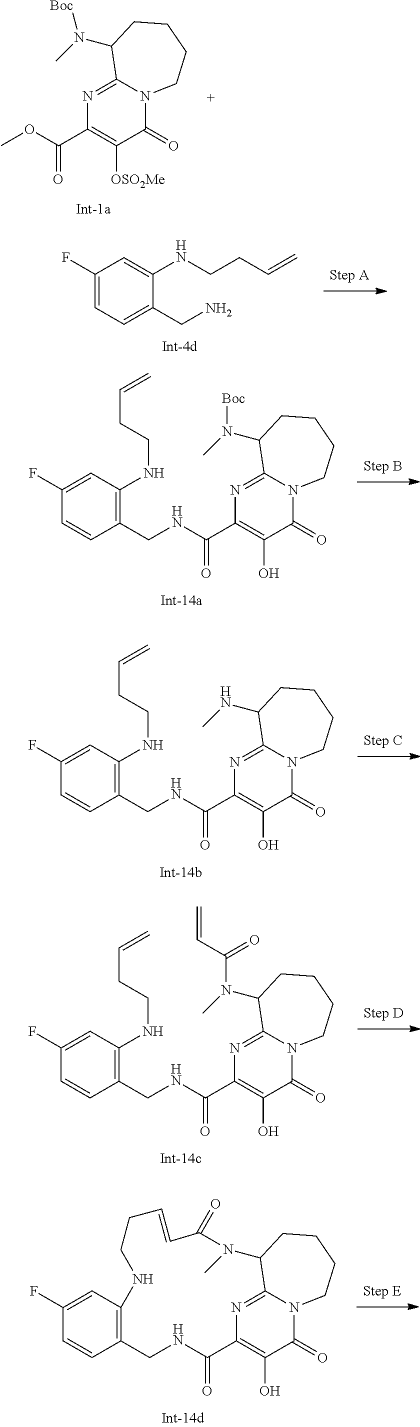

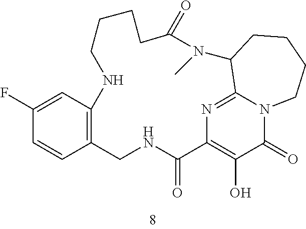 [SMILES: CN1C(=O)CCCCNc2cc(F)ccc2CNC(=O)c2nc3n(c(=O)c2O)CCCCC31]

### Step A—Synthesis of Intermediate Compound Int-14a

To a solution of compound Int-11 (148.4 mg, 0.33 mmol) in EtOH (3 mL) was added Int-4d (200 mg, 1 mmol) and the mixture was heated to 120° C. for 2 hours in a microwave. The reaction mixture was cooled to room temperature, concentrated in vacuo, and the resulting residue was purified using prep-TLC (10/1 dichloromethane/MeOH) to provide compound Int-14a (140 mg, 50%). LCMS anal. calcd. for C₂₇H₃₆FN₅O₅: 529.2. Found: 530.1 (M+1)⁺.

### Step B—Synthesis of Intermediate Compound Int-14b

To a solution of compound Int-14a (140 mg, 0.23 mmol) in dichloromethane (7 mL) was added TFA (7 mL) and the mixture was allowed to stir at room temperature for 2 hours. The mixture was concentrated in vacuo to provide compound Int-14b that was used directly in the next step. LCMS anal. calcd. for C₂₂H₂₈FN₅O₃: 429.2. Found: 430.1 (M+1)⁺.

### Step C—Synthesis of Intermediate Compound Int-14c

To a solution of compound Int-14b (100 mg, 0.19 mmol) and DIPEA (0.5 mL) in dichloromethane (5 mL) was added acryloyl chloride (18 mg, 0.19 mmol) in dichloromethane (1.0 mL) dropwise and the mixture was allowed to stir at room temperature for 2 hours. The mixture was diluted with dichloromethane (50 mL) and washed with a 1.0 M HCl solution and brine. The organic layer was dried over sodium sulfate, filtered, and concentrated in vacuo to provide compound Int-14c (100 mg, 90%). ¹H NMR (400 MHz, CDCl₃) δ 6.95-6.99 (m, 1H), 6.17-6.55 (m, 4H), 5.02-6.16 (m, 6H), 4.37-4.41 (m, 2H), 3.02-3.48 (m, 6H), 2.36-2.40 (m, 2H), 1.39-2.36 (m, 6H). LCMS anal. calcd. for C₂₅H₃₀FN₅O₄: 483.2. Found: 484.1 (M+1)⁺.

### Step D—Synthesis of Intermediate Compound Int-14d

A solution of compound Int-14c (60 mg, 0.12 mmol) in dichloromethane (60 mL) was degassed with nitrogen for 30 minutes. Zhan-1b catalyst (16.2 mg, 0.02 mmol) was added and the mixture was allowed to stir at room temperature for 20 hours under a nitrogen atmosphere. The mixture was concentrated in vacuo (bath temp<25° C.) and the residue obtained was purified using prep-TLC (10/1 dichloromethane/MeOH) to provide compound Int-14d (29 mg, 53%). ¹H NMR (400 MHz, CDCl₃) δ 7.13-7.17 (m, 1H), 6.42-6.49 (m, 2H), 5.51-5.56 (m, 1H), 5.41-5.47 (m, 1H), 4.37-4.39 (m, 1H), 4.21-4.34 (m, 1H), 3.37-3.62 (m, 4H), 2.88-3.15 (m, 4H), 2.13-2.24 (m, 2H), 1.43-1.49 (m, 6H). LCMS anal. calcd. for C₂₃H₂₆FN₅O₄: 455.2. Found: 456.1 (M+1)⁺.

### Step E—Synthesis of Compound 8

A mixture of compound Int-14d (29 mg, 0.12 mmol) and 10% Pd/C (8 mg) in MeOH (10 mL) was allowed to stir at room temperature for 2 hours under a hydrogen atmosphere. The mixture was filtered through celite and concentrated in vacuo. The crude residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 20-50%, 0-12 min] to provide Compound 8 (8 mg, 12%). ¹H NMR (400 MHz, CDCl₃) δ 7.12-7.19 (m, 1H), 6.42-6.51 (m, 2H), 5.75-5.82 (m, 1H), 5.28-5.36 (m, 1H), 4.39-4.51 (m, 2H), 3.48-3.54 (m, 2H), 3.32-3.40 (m, 2H), 2.94 (s, 3H), 2.51-2.72 (m, 8H), 1.17-2.03 (m, 3H). LCMS anal. calcd. for C₂₃H₂₈FN₅O₄: 457.2. Found: 458.1 (M+1)⁺.

### Example 15

### Preparation of Compound 9

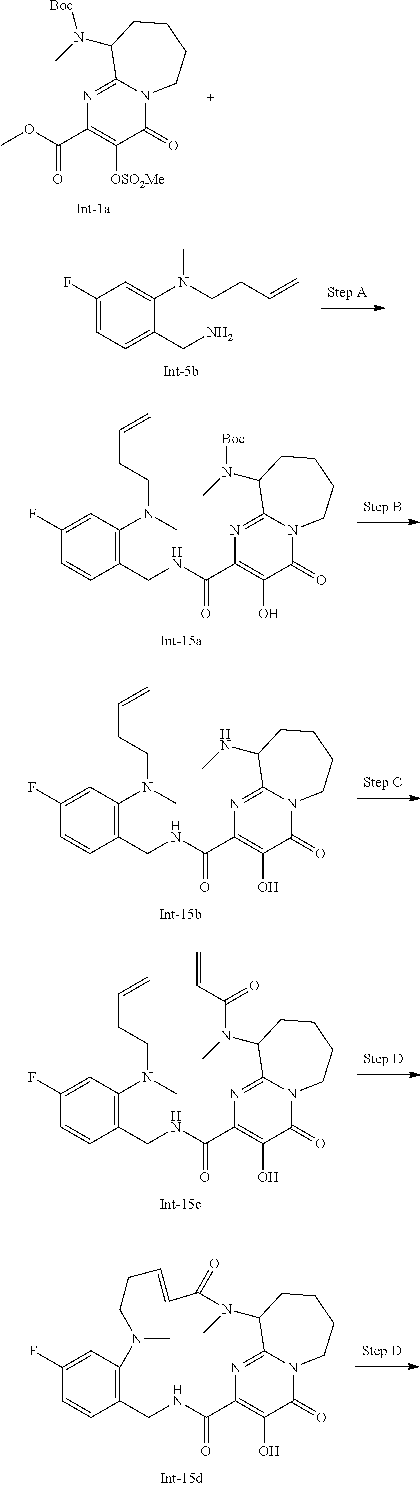

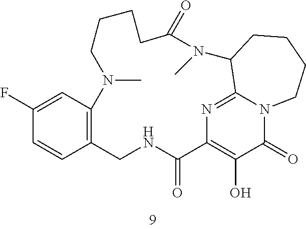 [SMILES: CN1CCCCC(=O)N(C)C2CCCCn3c2nc(c(O)c3=O)C(=O)NCc2ccc(F)cc21]

### Step A—Synthesis of Intermediate Compound Int-15a

A mixture of compound Int-1a (100 mg, 0.23 mmol) and Int-5d (150 mg, 0.70 mmol) in EtOH (2.0 mL) was heated to 120° C. for 2 hours in a microwave. The reaction mixture was cooled to room temperature, the solvent was concentrated in vacuo, and the residue obtained was purified using prep-TLC (20/1 dichloromethane/MeOH) to provide compound Int-15a (70 mg, 53%) as a yellow foam. ¹H NMR (400 MHz, CDCl₃) δ 7.19-7.23 (m, 1H), 6.58-6.62 (m, 2H), 5.85-5.91 (m, 2H), 5.13-5.21 (m, 3H), 4.61-4.64 (m, 1H), 4.43-4.49 (m, 1H), 4.01-4.08 (m, 2H), 3.48-3.52 (m, 1H), 3.07 (s, 3H), 2.51-2.54 (m, 2H), 1.97-2.03 (m, 3H), 1.53-1.58 (m, 6H), 1.47-1.51 (m, 9H). LCMS anal. calcd. for C₂₈H₃₈FN₅O₅: 543.2. Found: 544.1 (M+1)⁺.

### Step B—Synthesis of Intermediate Compound Int-15b

To a solution of compound Int-15a (70 mg, 0.13 mmol) in dichloromethane (3.0 mL) cooled to 0° C. was added TFA (0.5 mL). After warming to room temperature and stirring for 3 hours, the mixture was concentrated in vacuo to provide compound Int-15b (58 mg, 98%) as a yellow oil. LCMS anal. calcd. for C₂₃H₃₀FN₅O₃: 443.2. Found: 444.1 (M+1)⁺

### Step C—Synthesis of Intermediate Compound Int-15c

To a solution of compound Int-15b (57 mg, 0.13 mmol) and triethylamine (40 mg, 0.40 mmol) in dichloromethane (5 mL) was slowly added acryloyl chloride (18 mg, 0.20 mmol) in dichloromethane (1.0 mL) and the mixture was allowed to stir at room temperature for 6 hours. The reaction was diluted with dichloromethane (30 mL) then washed with 1.0 M HCl solution, brine and the organic layer was filtered and dried over anhydrous sodium sulfate. The solvent was removed in vacuo and the residue obtained was purified using prep-TLC (20/1 dichloromethane/MeOH) to provide compound Int-15c (37 mg, 52%) as a yellow foam. LCMS anal. calcd. for C₂₆H₃₂FN₅O₄: 497.2. Found: 498.1 (M+1)⁺.

### Step D—Synthesis of Intermediate Compound Int-15d

A solution of compound Int-15c (35 mg, 0.070 mmol) in dichloromethane (50 mL) was degassed with nitrogen for 30 minutes. Zhan-1b catalyst (20 mg, 0.028 mmol) was added and the mixture was allowed to stir at room temperature for 20 hours under a nitrogen atmosphere. The solvent was removed in vacuo and the resulting residue was purified using prep-TLC (15/1 dichloromethane/MeOH) to provide compound Int-15d (21 mg, 63%) as a yellow solid. LCMS anal. calcd. for C₂₄H₂₈FN₅O₄: 469.2. Found: 470.1 (M+1)⁺.

### Step E—Synthesis of Compound 9

A solution of compound Int-15d (20 mg, 0.043 mmol) in EtOAc (5.0 mL) was purged with nitrogen and 10% Pd/C (20 mg) was added. After stirring at room temperature for 2 hours under an atmosphere of hydrogen (balloon pressure), the mixture was filtered and washed with EtOAc (10 mL). The solvent was concentrated in vacuo and the resulting residue was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 43-63%, 0-12 min] to provide Compound 9 (8.0 mg, 39%) as a yellow solid. ¹H NMR (400 MHz, CDCl₃) δ 12.07 (br, 1H), 7.83-7.85 (br, 1H), 7.41-7.45 (m, 1H), 6.91-6.97 (m, 2H), 5.79-5.85 (m, 1H), 5.25-5.31 (m, 1H), 4.81-4.87 (m, 1H), 4.41-4.51 (m, 1H), 3.47-3.52 (m, 1H), 3.31-3.34 (m, 1H), 3.07 (s, 3H), 2.85-2.91 (m, 4H), 2.31-2.35 (m, 4H), 1.67-1.73 (m, 3H), 1.31-1.37 (m, 4H), 0.83-0.85 (m, 1H). LCMS anal. calcd. for C₂₄H₃₀FN₅O₄: 471.2. Found: 472.1 (M+1)⁺.

### Example 16

### Preparation of Compounds 10 and 11

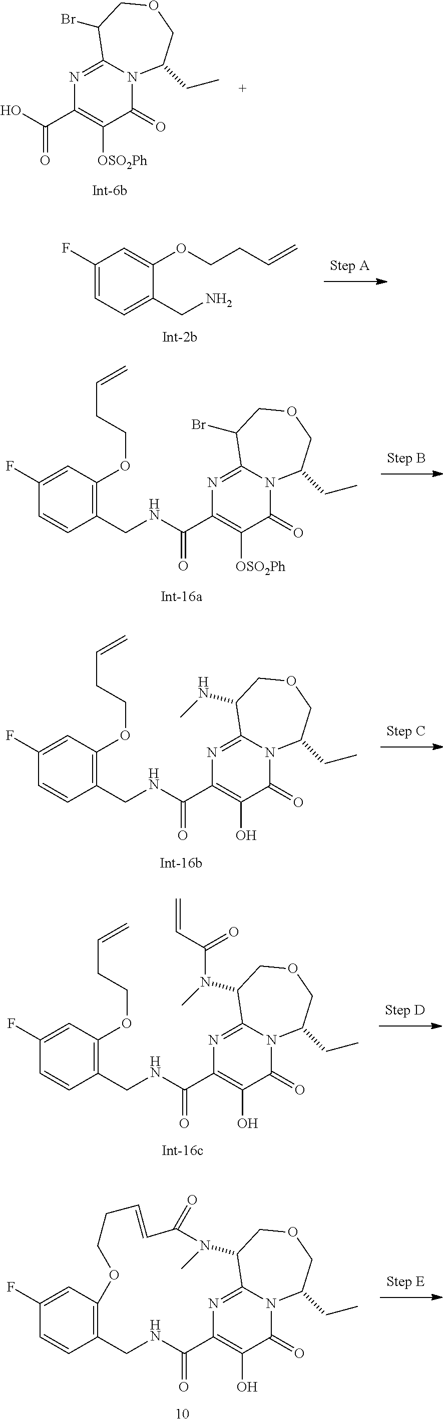

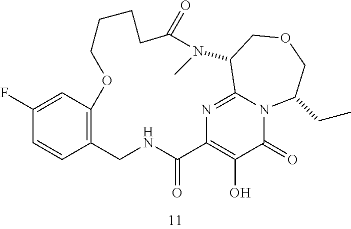 [SMILES: CC[C@H]1COC[C@@H]2c3nc(c(O)c(=O)n31)C(=O)NCc1ccc(F)cc1OCCCCC(=O)N2C]

### Step A—Synthesis of Intermediate Compound Int-16a

To a mixture of compound Int-6b (384 mg, 0.82 mmol), Int-2b (481 mg, 2.5 mmol), and hunig's base (254 mg, 2.0 mmol) in DMF (8.0 mL) cooled to 0° C. was added PyClu (672 mg, 2.0 mmol) and the mixture was allowed to stir at room temperature for 1 hour. The reaction mixture was diluted with water and extracted with EtOAc. The organic layer was washed with water and brine then dried over sodium sulfate. The solvent was removed in vacuo and the residue obtained was purified using silica gel chromatography (50/1 dichloromethane/CH₃OH) to provide compound Int-16a (265 mg, 52%) as a yellow foam. LCMS anal. calcd. for C₂₈H₂₉BrFN₃O₇S: 649.1. Found: 650.2 (M+1)⁺.

### Step B—Synthesis of Intermediate Compound Int-16b

To a solution of compound Int-16a (265 mg, 0.41 mmol) in methanol (4.0 mL) was added CH₃NH₂ solution (1.5 mL, 33% solution in MeOH) and the mixture was allowed to stir at room temperature for about 15 hours. The solvent was removed in vacuo to provide compound Int-16b (171 mg, 91%) as an oil. ¹H NMR (400 MHz, CDCl₃) δ 7.76-7.81 (m, 1H), 7.29-7.38 (m, 1H), 6.65-6.69 (m, 1H), 6.21-6.40 (br, 1H), 5.98-6.06 (m, 1H), 5.17-5.23 (m, 2H), 4.57 (s, 2H), 4.31-4.40 (m, 1H), 3.91-4.10 (m, 6H), 2.95 (s, 3H), 2.71-2.80 (m, 1H), 2.31-2.37 (m, 2H), 1.71-1.79 (m, 2H), 1.05-1.08 (t, J=7.2 Hz, 3H). LCMS anal. calcd. for C₂₃H₂₉FN₄O₅: 460.2. Found: 461.1 (M+1)⁺.

### Step C—Synthesis of Intermediate Compound Int-16c

To a mixture of compound Int-16b (170 mg, 0.37 mmol) and Et₃N (101 mg, 1.0 mmol) in dichloromethane (5 mL) cooled to 0° C. was added dropwise a solution of acryloyl chloride (0.40 mL, 1.0 M in dichloromethane, 0.40 mmol) under a nitrogen atmosphere. After addition, the reaction mixture was allowed to stir at room temperature for 2 hours. The reaction mixture was diluted with dichloromethane and washed with 1.0 M HCl solution and brine. The mixture was then dried over sodium sulfate, filtered, and concentrated in vacuo to provide compound Int-16c (160 mg, 87%) as an oil. LCMS anal. calcd. for C₂₆H₃₁FN₄O₆: 514.1. Found: 515.2 (M+1)⁺.

### Step D—Synthesis of Compound 10

A solution of compound Int-16c (160 mg, 0.31 mmol) in dichloromethane (80 mL) was degassed with nitrogen for 30 minutes. Zhan-1B catalyst (30 mg, 0.042 mmol) was then added and the mixture was allowed to stir at room temperature for about 15 hours. The solvent was removed in vacuo and the residue obtained was purified using prep-TLC (20/1 dichloromethane/CH₃OH) to provide Compound 10 (100 mg, 51%) as a yellow solid. ¹H NMR (400 MHz, CDCl₃) δ 7.85-7.86 (m, 1H), 7.42-7.51 (m, 2H), 6.95-7.08 (m, 1H), 6.56-6.70 (m, 3H), 6.32-6.41 (m, 1H), 5.20-5.23 (m, 1H), 4.61-4.64 (m, 2H), 3.54 (s, 3H), 2.94 (s, 3H), 2.09-2.11 (m, 3H), 4.55 (s, 2H), 4.05-4.28 (m, 7H), 3.41 (s, 1H), 2.72-2.98 (m, 4H), 1.81-1.92 (m, 2H), 1.11-1.18 (m, 3H). LCMS anal. calcd. for C₂₄H₂₇FN₄O₆: 486.2. Found: 487.2 (M+1)⁺.

### Step E—Synthesis of Compound 11

A solution of Compound 10 (90 mg, 0.21 mmol) in CH₃OH (10 mL) was purged with nitrogen and 10% Pd/C (50 mg) was added. The mixture was allowed to stir over a hydrogen atmosphere under balloon pressure for 2 hours. The mixture was filtered and concentrated in vacuo and the residue obtained was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 30-60%, 0-15 min] to provide Compound 11 (25 mg, 21%) as an off-white solid. ¹H NMR (400 MHz, CDCl₃) δ 7.75-7.82 (m, 1H), 7.31-7.40 (m, 1H), 6.61-6.71 (m, 1H), 6.21-6.40 (br, 1H), 4.57 (s, 2H), 4.31-4.40 (m, 1H), 3.91-4.10 (m, 6H), 2.98 (s, 3H), 2.71-2.80 (m, 1H), 2.31-2.41 (m, 3H), 1.91-2.03 (m, 4H), 1.05-1.08 (t, J=7.2 Hz, 3H). LCMS anal. calcd. for C₂₄H₂₉FN₄O₆: 488.2. Found: 489.2 (M+1)⁺.

### Example 17

### Preparation of Compounds 12 and 13

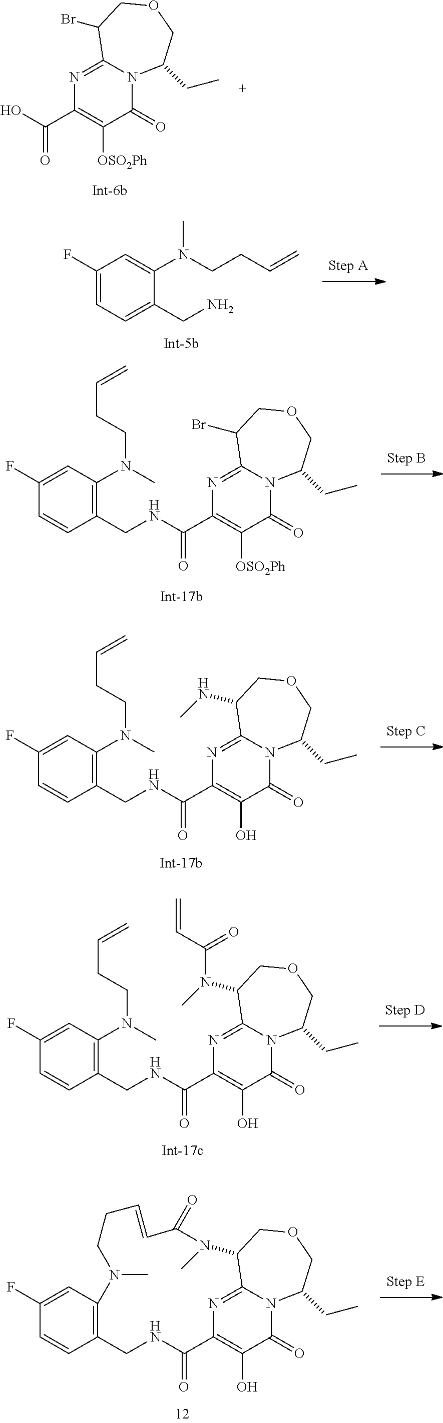

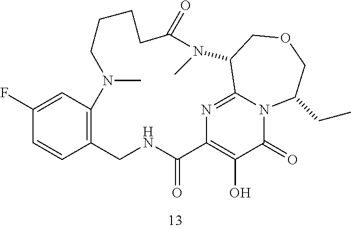 [SMILES: CC[C@H]1COC[C@@H]2c3nc(c(O)c(=O)n31)C(=O)NCc1ccc(F)cc1N(C)CCCCC(=O)N2C]

### Step A—Synthesis of Intermediate Compound Int-17a

To a mixture of compound Int-6b (150 mg, 1.05 mmol), Int-5b (630 mg, 3.2 mmol), and DIPEA (320 mg, 2.6 mmol) in DMF (10 mL) cooled to 0° C. was added PyClu (881 mg, 2.6 mmol) and the mixture was allowed to stir at room temperature for 1 hour. The reaction mixture was diluted with water and extracted with EtOAc. The organic layer was washed with water and brine then dried over sodium sulfate. The solvent was removed in vacuo and the residue obtained was purified using silica gel chromatography (50/1 dichloromethane/CH₃OH) to provide compound Int-17a (70 mg, 37%) as a yellow foam. LCMS anal. calcd. for C₂₉H₃₂BrFN₄O₆S: 662.1. Found: 663.2 (M+1)⁺.

### Step B—Synthesis of Intermediate Compound Int-17b

To a solution of compound Int-17a (120 mg, 0.18 mmol) in methanol (3 mL) was added CH₃NH₂ solution (1 mL, 33% solution in MeOH) and the mixture was allowed to stir at room temperature for about 15 hours. The solvent was removed in vacuo to provide compound Int-17b (70 mg, 83%) as an oil. ¹H NMR (400 MHz, MeOD) δ 7.41-7.46 (m, 1H), 7.13-7.18 (m, 1H), 6.83-6.87 (m, 1H), 5.97-6.01 (m, 1H), 5.17-5.23 (m, 2H), 4.49-4.68 (m, 3H), 4.29-4.38 (m, 1H), 3.94-4.03 (m, 3H), 2.92-3.19 (m, 9H), 2.31-2.38 (m, 2H), 1.51-1.61 (m, 2H), 1.12-1.15 (t, J=7.2 Hz, 3H). LCMS anal. calcd. for C₂₄H₃₂FN₅O₄: 473.2. Found: 474.1 (M+1)⁺.

### Step C—Synthesis of Intermediate Compound Int-17c

To a mixture of compound Int-17b (70 mg, 0.15 mmol) and Et₃N (55 mg, 0.54 mmol) in dichloromethane (5 mL) cooled to 0° C. was added dropwise a solution of acryloyl chloride (0.20 mL, 1.0 M in dichloromethane, 0.20 mmol) under a nitrogen atmosphere. After addition, the reaction mixture was allowed to stir at room temperature for 2 hours. The reaction mixture was diluted with dichloromethane and washed with 1.0 M HCl solution and brine. The mixture was then dried over sodium sulfate, filtered, and concentrated in vacuo to provide compound Int-17c (65 mg, 81%) as an oil. LCMS anal. calcd. for C₂₇H₃₄FN₅O₅: 527.2. Found: 528.1 (M+1)⁺.

### Step D—Synthesis of Compound 12

A solution of compound Int-17c (65 mg, 0.12 mmol) in dichloromethane (50 mL) was degassed with nitrogen for 30 minutes. Zhan-1B catalyst (20 mg, 0.025 mmol) was then added and the mixture was allowed to stir at room temperature for about 15 hours. The solvent was removed in vacuo and the residue obtained was purified using prep-TLC (20/1 dichloromethane/CH₃OH) to provide Compound 12 (40 mg, 62%) as a yellow solid. ¹H NMR (400 MHz, MeOD) δ 7.45-7.52 (m, 1H), 6.82-6.99 (m, 3H), 6.51-6.58 (m, 0.5H), 6.16-6.25 (m, 0.5H), 4.37-4.71 (m, 5H), 4.01-4.21 (m, 2H), 3.08-3.18 (m, 2H), 2.51-2.78 (m, 9H), 1.91-2.11 (m, 2H), 1.11-1.18 (m, 3H). LCMS anal. calcd. for C₂₅H₃₀FN₅O₅: 499.1. Found: 500.1 (M+1)⁺.

### Step E—Synthesis of Compound 13

A solution of Compound 12 (35 mg, 0.071 mmol) in CH₃OH (16 mL) was purged with nitrogen and 10% Pd/C (50 mg) was added. The mixture was allowed to stir over a hydrogen atmosphere under balloon pressure for 2 hours. The mixture was filtered and concentrated in vacuo and the residue obtained was purified using prep-HPLC [Column-type: YMC-pack ODS-AQ (150 mm×30 mm, 5 μm). Mobile phase A: water (containing 0.075% TFA, V/V), Mobile phase B: acetonitrile, Flow rate: 25 mL/min, Gradient: 30-60%, 0-18 min] to provide Compound 13 (10 mg, 29%) as an off-white solid. ¹H NMR (400 MHz, MeOD) δ 7.43-7.51 (m, 1H), 7.14-7.20 (m, 1H), 6.85-6.91 (m, 1H), 4.51-4.70 (m, 3H), 4.31-4.41 (m, 1H), 3.95-4.05 (m, 3H), 2.95-3.21 (m, 6H), 2.71-2.81 (m, 4H), 2.51-2.61 (m, 1H), 2.31-2.42 (m, 1H), 2.15-2.26 (m, 1H), 1.95-2.05 (m, 1H), 1.52-1.85 (m, 4H), 1.12-1.15 (t, J=7.2 Hz, 3H). LCMS anal. calcd. for C₂₅H₃₂FN₅O₅: 501.2. Found: 502.1 (M+1)⁺.

Compounds 14-28, set forth in the table below, were made using the methods described in the Examples above.

| No. | Structure | MS |
|---|---|---|
| 14 | 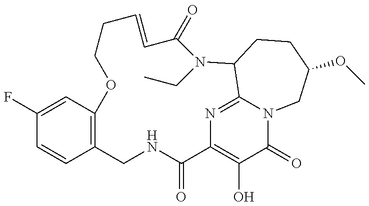 [SMILES: CCN1C(=O)/C=C/CCOc2cc(F)ccc2CNC(=O)c2nc3n(c(=O)c2O)C[C@@H](OC)CCC31] | 501 (M + H) |
| 15 | 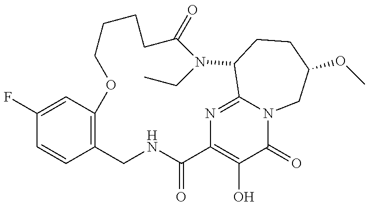 | 503 (M + H) |
| 16 | 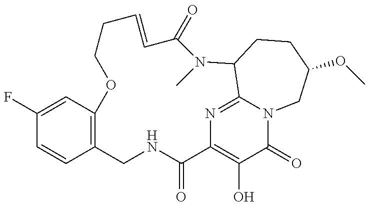 [SMILES: CO[C@H]1CCC2c3nc(c(O)c(=O)n3C1)C(=O)NCc1ccc(F)cc1OCC/C=C/C(=O)N2C] | 487 (M + H) |
| 17 | 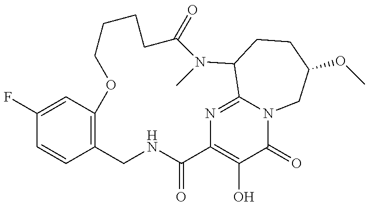 [SMILES: CO[C@H]1CCC2c3nc(c(O)c(=O)n3C1)C(=O)NCc1ccc(F)cc1OCCCCC(=O)N2C] | 489 (M + H) |
| 18 | 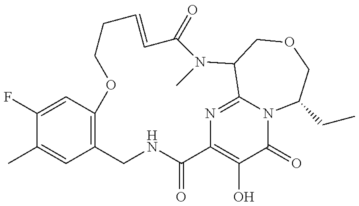 [SMILES: CC[C@H]1COCC2c3nc(c(O)c(=O)n31)C(=O)NCc1cc(C)c(F)cc1OCC/C=C/C(=O)N2C] | 501 (M + H) |
| 19 | 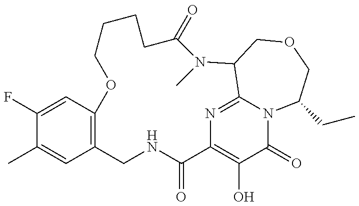 [SMILES: CC[C@H]1COCC2c3nc(c(O)c(=O)n31)C(=O)NCc1cc(C)c(F)cc1OCCCCC(=O)N2C] | 503 (M + H) |
| 20 | 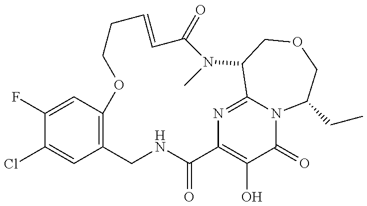 | 521 (M + H) |
| 21 | 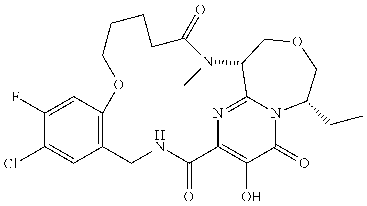 | 523 (M + H) |
| 22 | 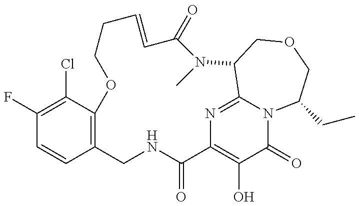 | 521 (M + H) |
| 23 | 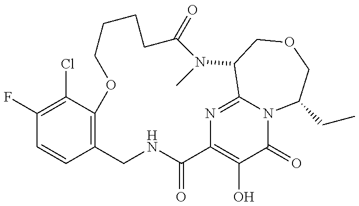 | 523 (M + H) |
| 24 | 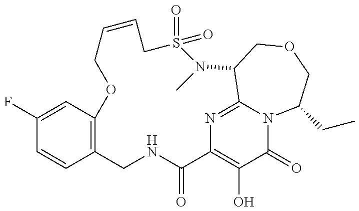 | 523 (M + H) |
| 25 | 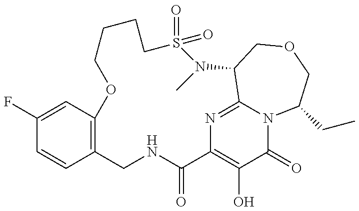 [SMILES: CC[C@H]1COC[C@@H]2c3nc(c(O)c(=O)n31)C(=O)NCc1ccc(F)cc1OCCCCS(=O)(=O)N2C] | 525 (M + H) |
| 26 | 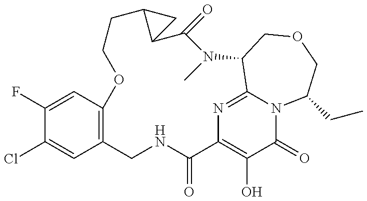 [SMILES: CC[C@H]1COC[C@@H]2c3nc(c(O)c(=O)n31)C(=O)NCc1cc(Cl)c(F)cc1OCCC1CC1C(=O)N2C] | 535 (M + H) |
| 27 | 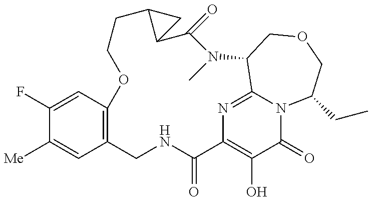 [SMILES: CC[C@H]1COC[C@@H]2c3nc(c(O)c(=O)n31)C(=O)NCc1cc(C)c(F)cc1OCCC1CC1C(=O)N2C] | 515 (M + H) |
| 28 | 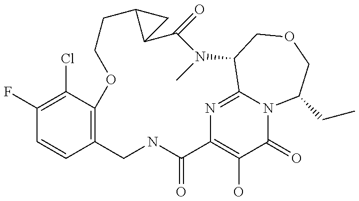 [SMILES: CC[C@H]1COC[C@@H]2c3nc(c(O)c(=O)n31)C(=O)NCc1ccc(F)c(Cl)c1OCCC1CC1C(=O)N2C] | 535 (M + H) |

### Example 18

### In Vitro Inhibition of HIV Replication

Assays for the inhibition of acute HIV-1 infection of T-lymphoid cells were conducted in accordance with Vacca, J. P. et al., Proc. Natl. Acad. Sci. USA 1994, 91: 4096. Representative compounds of the present invention exhibit inhibition of HIV replication in this assay (also referred to herein as the “spread assay”). Data for selected compounds of the present invention, obtained using this method, is provided in the table below. IC₉₅ data was obtained in the presence of 10% NHS.

| | IC₉₅ |
|---|---|
| Compound | (nM) |
| 2 | 179 |
| 3 | 2262 |
| 4 | 86 |
| 5 | 507 |
| 6 | 387 |
| 7 | 577 |
| 8 | 208 |
| 9 | 80 |
| 10 | 12 |
| 11 | 9 |
| 12 | 43 |
| 13 | 15 |
| 14 | 71 |
| 15 | 65 |
| 16 | 26 |
| 17 | 42 |
| 18 | 6 |
| 19 | 9 |
| 22 | 75 |
| 23 | 10 |
| 24 | 39 |
| 25 | 68 |

### Uses of the Macrocyclic Compounds

The Macrocyclic Compounds are useful in human and veterinary medicine for treating or preventing HIV infection in a subject. In one embodiment, the Macrocyclic Compounds can be inhibitors of HIV viral replication. In a specific embodiment, the Macrocyclic Compounds are inhibitors of HIV-1. Accordingly, the Macrocyclic Compounds are useful for treating HIV infections and AIDS. In accordance with the invention, the Macrocyclic Compounds can be administered to a subject in need of treatment or prevention of HIV infection.

Accordingly, in one embodiment, the invention provides methods for treating HIV infection in a subject comprising administering to the subject an effective amount of at least one Macrocyclic Compound or a pharmaceutically acceptable salt thereof. In a specific embodiment, the present invention provides methods for treating AIDS in a subject comprising administering to the subject an effective amount of at least one Macrocyclic Compound or a pharmaceutically acceptable salt thereof.

### Treatment or Prevention of HIV Infection

The Macrocyclic Compounds are useful in the inhibition of HIV, the treatment of HIV infection and/or reduction of the likelihood or severity of symptoms of HIV infection and the inhibition of HIV viral replication and/or HIV viral production in a cell-based system. For example, the Macrocyclic Compounds are useful in treating infection by HIV after suspected past exposure to HIV by such means as blood transfusion, exchange of body fluids, bites, accidental needle stick, or exposure to subject blood during surgery or other medical procedures.

In one embodiment, the HIV infection has progressed to AIDS.

Accordingly, in one embodiment, the invention provides methods for treating HIV infection in a subject, the methods comprising administering to the subject an effective amount of at least one Macrocyclic Compound or a pharmaceutically acceptable salt thereof. In a specific embodiment, the amount administered is effective to treat or prevent infection by HIV in the subject. In another specific embodiment, the amount administered is effective to inhibit HIV viral replication and/or viral production in the subject.

The Macrocyclic Compounds are also useful in the preparation and execution of screening assays for antiviral compounds. For example the Macrocyclic Compounds are useful for identifying resistant HIV cell lines harboring mutations, which are excellent screening tools for more powerful antiviral compounds. Furthermore, the Macrocyclic Compounds are useful in establishing or determining the binding site of other antivirals to the HIV Integrase.

The compositions and combinations of the present invention can be useful for treating a subject suffering from infection related to any HIV genotype.

### Combination Therapy

In another embodiment, the present methods for treating or preventing HIV infection can further comprise the administration of one or more additional therapeutic agents which are not Macrocyclic Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a subject, the method comprising administering to the subject: (i) at least one Macrocyclic Compound (which may include two or more different Macrocyclic Compounds), or a pharmaceutically acceptable salt thereof, and (ii) at least one additional therapeutic agent that is other than a Macrocyclic Compound, wherein the amounts administered are together effective to treat or prevent a viral infection.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Macrocyclic Compound and an additional therapeutic agent may be present in fixed amounts (dosage amounts) in a single dosage unit (e.g., a capsule, a tablet and the like).

In one embodiment, the at least one Macrocyclic Compound is administered during a time when the additional therapeutic agent(s) exert their prophylactic or therapeutic effect, or vice versa.

In another embodiment, the at least one Macrocyclic Compound and the additional therapeutic agent(s) are administered in doses commonly employed when such agents are used as monotherapy for treating a viral infection.

In another embodiment, the at least one Macrocyclic Compound and the additional therapeutic agent(s) are administered in doses lower than the doses commonly employed when such agents are used as monotherapy for treating a viral infection.

In still another embodiment, the at least one Macrocyclic Compound and the additional therapeutic agent(s) act synergistically and are administered in doses lower than the doses commonly employed when such agents are used as monotherapy for treating a viral infection.

In one embodiment, the at least one Macrocyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HIV infection.

In another embodiment, the viral infection is AIDS.

The at least one Macrocyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Macrocyclic Compound and the additional therapeutic agent(s) may inhibit the resistance of a viral infection to these agents.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| Dolutegravir | PI |
| efavirenz, EFV, Sustiva ®, Stocrin ® | nnRTI |
| efavirenz + emtricitabine + tenofovir DF, Atripla ® | nnRTI + |
| | nRTI |
| Elvitegravir | InI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ® | InI |
| rilpivirine, TMC-278 | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor. Some of the drugs listed in the table are used in a salt form; e.g., abacavir sulfate, indinavir sulfate, atazanavir sulfate, nelfinavir mesylate.

In one embodiment, the one or more anti-HIV drugs are selected from raltegravir, lamivudine, abacavir, ritonavir, dolutegravir, darunavir, atazanavir, emtricitabine, tenofovir, elvitegravir, rilpivirine and lopinavir.

In another embodiment, the compound of formula (I) is used in combination with a single anti-HIV drug which is raltegravir.

In another embodiment, the compound of formula (I) is used in combination with a single anti-HIV drug which is lamivudine.

In still another embodiment, the compound of formula (I) is used in combination with a single anti-HIV drug which is atazanavir.

In another embodiment, the compound of formula (I) is used in combination with a single anti-HIV drug which is darunavir.

In another embodiment, the compound of formula (I) is used in combination with a single anti-HIV drug which is rilpivirine.

In yet another embodiment, the compound of formula (I) is used in combination with a single anti-HIV drug which is dolutegravir.

In another embodiment, the compound of formula (I) is used in combination with a single anti-HIV drug which is elvitegravir.

In one embodiment, the compound of formula (I) is used in combination with two anti-HIV drugs which are lamivudine and abacavir.

In another embodiment, the compound of formula (I) is used in combination with two anti-HIV drugs which are darunavir and raltegravir.

In another embodiment, the compound of formula (I) is used in combination with two anti-HIV drugs which are emtricitabine and tenofovir.

In still another embodiment, the compound of formula (I) is used in combination with two anti-HIV drugs which are atazanavir and raltegravir.

In another embodiment, the compound of formula (I) is used in combination with two anti-HIV drugs which are ritonavir and lopinavir.

In another embodiment, the compound of formula (I) is used in combination with two anti-HIV drugs which are lamivudine and raltegravir.

In one embodiment, the compound of formula (I) is used in combination with three anti-HIV drug which are abacavir, lamivudine and raltegravir.

In another embodiment, the compound of formula (I) is used in combination with three anti-HIV drug which are lopinavir, ritonavir and raltegravir.

In one embodiment, the present invention provides pharmaceutical compositions comprising (i) a compound of formula (I) or a pharmaceutically acceptable salt thereof; (ii) a pharmaceutically acceptable carrier; and (iii) one or more additional anti-HIV agents selected from lamivudine, abacavir, ritonavir and lopinavir, or a pharmaceutically acceptable salt thereof, wherein the amounts present of components (i) and (iii) are together effective for the treatment or prophylaxis of infection by HIV or for the treatment, prophylaxis, or delay in the onset or progression of AIDS in the subject in need thereof.

In another embodiment, the present invention provides a method for the treatment or prophylaxis of infection by HIV or for the treatment, prophylaxis, or delay in the onset or progression of AIDS in a subject in need thereof, which comprises administering to the subject (i) a compound of formula (I) or a pharmaceutically acceptable salt thereof and (ii) one or more additional anti-HIV agents selected from lamivudine, abacavir, ritonavir and lopinavir, or a pharmaceutically acceptable salt thereof, wherein the amounts administered of components (i) and (ii) are together effective for the treatment or prophylaxis of infection by HIV or for the treatment, prophylaxis, or delay in the onset or progression of AIDS in the subject in need thereof.

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and the like. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The doses and dosage regimen of the other agents used in the combination therapies of the present invention for the treatment or prevention of HIV infection can be determined by the attending clinician, taking into consideration the approved doses and dosage regimen in the package insert; the age, sex and general health of the subject; and the type and severity of the viral infection or related disease or disorder. When administered in combination, the Macrocyclic Compound(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

### Compositions and Administration

When administered to a subject, the Macrocyclic Compounds can be administered as a component of a composition that comprises a pharmaceutically acceptable carrier or vehicle. The present invention provides pharmaceutical compositions comprising an effective amount of at least one Macrocyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Macrocyclic Compounds are administered orally.

In another embodiment, the one or more Macrocyclic Compounds are administered intravenously.

In one embodiment, a pharmaceutical preparation comprising at least one Macrocyclic Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Macrocyclic Compound(s) by weight or volume. In various embodiments, the present compositions can contain, in one embodiment, from about 1% to about 70% or from about 5% to about 60% of the Macrocyclic Compound(s) by weight or volume.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Macrocyclic Compounds will be regulated according to the judgment of the attending clinician considering such factors as age, condition and size of the subject as well as severity of the symptoms being treated. The compositions of the invention can further comprise one or more additional therapeutic agents, selected from those listed above herein. Accordingly, in one embodiment, the present invention provides compositions comprising: (i) at least one Macrocyclic Compound or a pharmaceutically acceptable salt thereof; (ii) one or more additional therapeutic agents that are not a Macrocyclic Compound; and (iii) a pharmaceutically acceptable carrier, wherein the amounts in the composition are together effective to treat HIV infection.

### Kits

In one aspect, the present invention provides a kit comprising a therapeutically effective amount of at least one Macrocyclic Compound, or a pharmaceutically acceptable salt or prodrug of said compound and a pharmaceutically acceptable carrier, vehicle or diluent.

In another aspect the present invention provides a kit comprising an amount of at least one Macrocyclic Compound, or a pharmaceutically acceptable salt or prodrug of said compound and an amount of at least one additional therapeutic agent listed above, wherein the amounts of the two or more active ingredients result in a desired therapeutic effect. In one embodiment, the one or more Macrocyclic Compounds and the one or more additional therapeutic agents are provided in the same container. In one embodiment, the one or more Macrocyclic Compounds and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 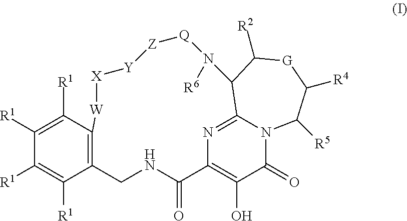
or a pharmaceutically acceptable salt thereof,
wherein:
G is —O— or —CH(R³)—
Q is —C(O)— or —S(O)₂—;
W is a bond, —O— or —N(R⁸)—;
X is a bond or —C(O)—;
Y is C₃-C₅ alkylene or C₃-C₅ alkenylene;
Z is a bond, —O— or —N(R⁸)C(O)—;
each occurrence of R¹ is independently selected from H, C₁-C₆ alkyl, halo, C₁-C₆ haloalkyl, 3 to 7-membered cycloalkyl, —OR⁷, —N(R⁷)₂, —CN, —C(O)R⁷, —C(O)OR⁷, —C(O)N(R⁷)₂ and —NHC(O)R⁷;
R², R³, R⁴ and R⁵ are each independently selected from H, C₁-C₆ alkyl, halo, —OR⁷ and —N(R⁷)₂;
R⁶ is H or C₁-C₆ alkyl, 3 to 7-membered cycloalkyl, C₆-C₁₀ aryl, 4 to 7-membered cycloalkyl or 5 or 6-membered monocyclic heteroaryl;
each occurrence of R⁷ is independently selected from C₁-C₆ alkyl, 3 to 7-membered cycloalkyl, C₆-C₁₀ aryl, 4 to 7-membered cycloalkyl or 5 or 6-membered monocyclic heteroaryl; and
each occurrence of R⁸ is independently selected from H and C₁-C₆ alkyl.

2. The compound of claim 1, wherein R⁶ is methyl.

3. The compound of claim 1, wherein G is —O— or —CH₂—.

4. The compound of claim 1, wherein R⁵ is H or C₁-C₆ alkyl.

5. The compound of claim 1, wherein R¹ is a single halo substituent.

6. The compound of claim 1, having the formula: 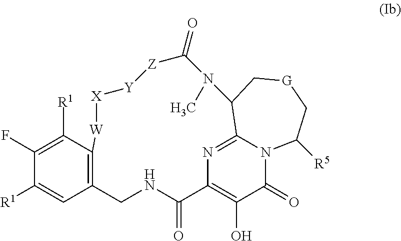
or a pharmaceutically acceptable salt thereof,
wherein:
G is —O— or —CH₂—
W is a bond, —O— or —N(R⁸)C(O)—;
X is a bond or —C(O)—;
Y is C₃-C₅ alkylene or C₃-C₅ alkenylene;
Z is a bond, —O— or —C(O)—;
R¹ is H or halo; and
R⁵ is H or C₁-C₆ alkyl.

7. The compound of claim 6, wherein G is —CH₂— and R⁵ is H.

8. The compound of claim 6, wherein G is O and R⁵ is ethyl.

9. The compound of claim 6, wherein W is —O— or —N(R⁸)—, X is a bond and Z is a bond.

10. The compound of claim 6, wherein W, X and Z are each a bond.

11. The compound of claim 6, wherein W is —N(R⁸)—, X is —C(O)— and Z is a bond.

12. The compound of claim 6, wherein Z is —O— or —N(R⁸)C(O)— and W and X are each a bond.

13. The compound of claim 1 having the structure: 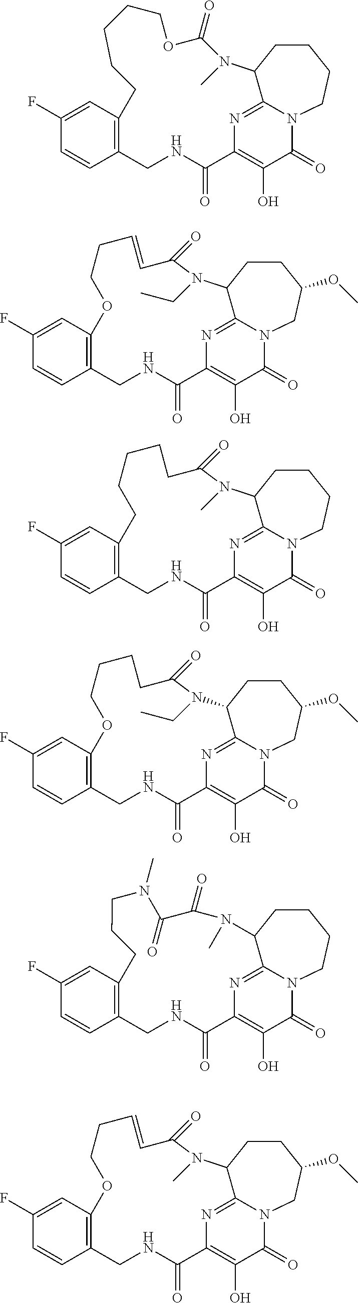 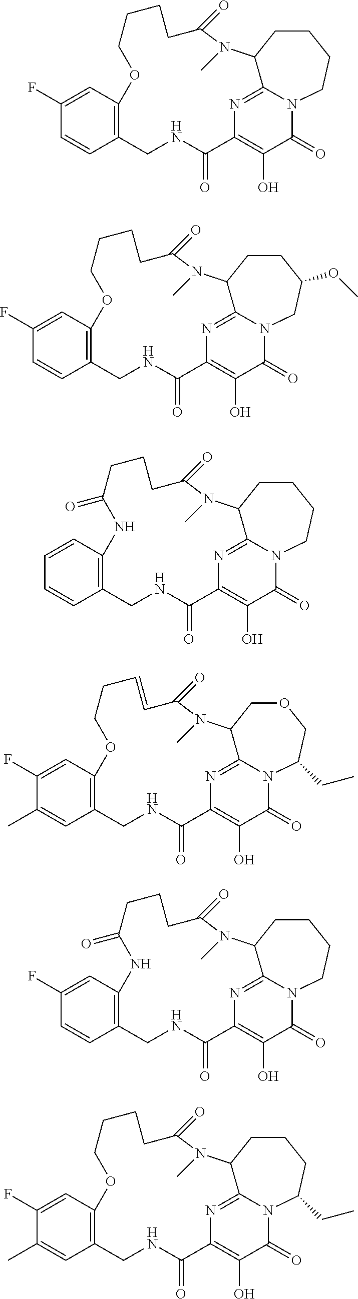 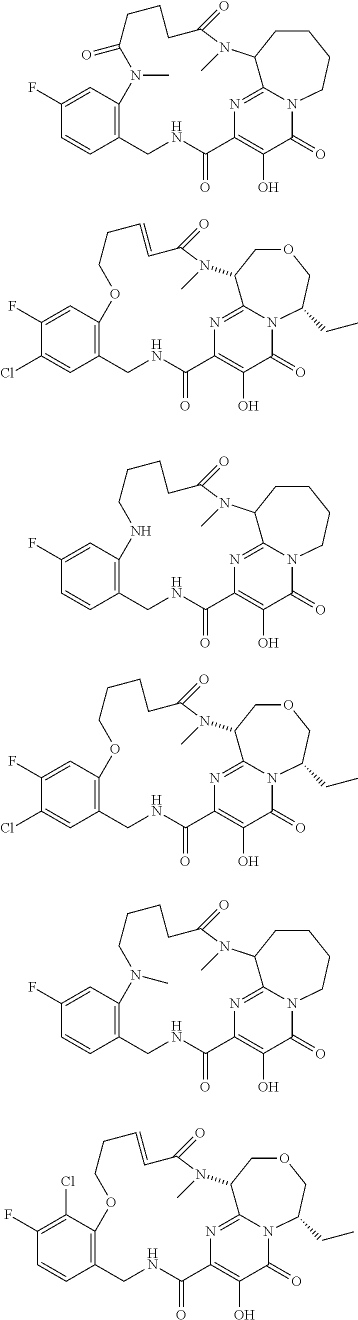 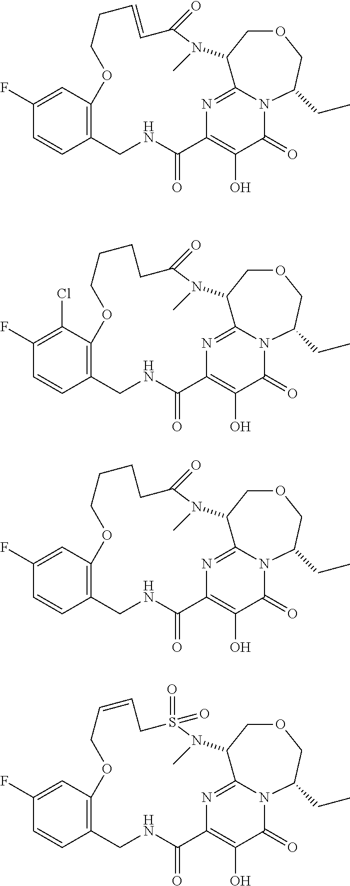 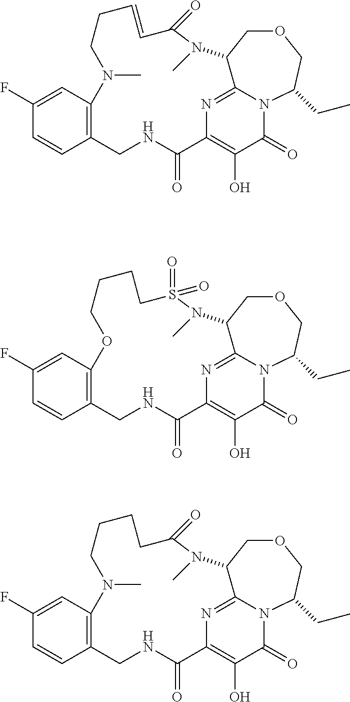
or a pharmaceutically acceptable salt thereof.

14. A pharmaceutical composition comprising an effective amount of a compound according to any one of claims 1 to 13, or a pharmaceutically acceptable salt thereof, and a pharmaceutically acceptable carrier.

15. A method for the inhibition of HIV integrase in a subject in need thereof which comprises administering to the subject an effective amount of the compound according to any one of claims 1 to 13, or a pharmaceutically acceptable salt thereof.

16. A method for the treatment of infection by HIV or for the treatment, or delay in the onset or progression of AIDS in a subject in need thereof, which comprises administering to the subject an effective amount of the compound according to any one of claims 1 to 13, or a pharmaceutically acceptable salt thereof.

17. (canceled)

18. (canceled)

19. The composition of claim 14, further comprising one or more additional therapeutic agents selected from raltegravir, lamivudine, abacavir, ritonavir, dolutegravir, arunavir, atazanavir, emtricitabine, tenofovir, elvitegravir, rilpivirine and lopinavir.

20. The method of claim 16, further comprising administering to the subject one or more additional therapeutic agents selected from raltegravir, abacavir, lamivudine, ritonavir and lopinavir, wherein the amounts administered of the compound of any one of claims 1-13 and the one or more additional therapeutic agents, are together effective to treat infection by HIV or to treat or delay the onset or progression of AIDS.
